(19) United States
(12) Patent Application Publication
(10) Pub. No.: US 2025/0270660 A1
(43) Pub. Date: Aug. 28, 2025

(54) ASSAY FOR T CELL DEPENDENT MULTISPECIFIC COMPOUNDS

(71) Applicant: LAVA THERAPEUTICS N.V., Utrecht (NL)

(72) Inventors: Thilo Alexander RIEDL, Utrecht (NL); Kim HOENDERDOS, Utrecht (NL); Anton Egbert Peter ADANG, Utrecht (NL); Johannes Jelle VAN DER VLIET, Utrecht (NL)

(21) Appl. No.: 18/989,870

(22) Filed: Dec. 20, 2024

Related U.S. Application Data
(63) Continuation of application No. PCT/US2023/068989, filed on Jun. 23, 2023.
(60) Provisional application No. 63/354,913, filed on Jun. 23, 2022.

(51) Int. Cl. C12Q 1/6897; G01N 33/569; G01N 33/574

(52) U.S. Cl. CPC C12Q 1/6897; G01N 33/56972; G01N 33/57484

(57) ABSTRACT

The present disclosure provides reporter T cells expressing a γδ T cell receptor (TCR) and a reporter gene, wherein the reporter gene is expressed when the T cell is activated. Also provided are assays that utilize the modified T cells to detect and/or quantitate T cell dependent binding compounds directed to a γδTCR and a target antigen and to determine the relative potency of such binding compounds.

### CROSS-REFERENCE TO RELATED APPLICATIONS

The present application claims priority to U.S. Provisional Patent Application No. 63/354,913, filed on Jun. 23, 2022, the content of which is incorporated herein by reference in its entirety.

### ELECTRONIC VERSION OF THE SEQUENCE LISTING

The contents of the electronic sequence listing (LVAT_023_01WO_SeqList_ST26.xml; Size: 140,393 bytes; and Date of Creation: Jun. 23, 2023) are herein incorporated by reference in its entirety.

### FIELD OF THE DISCLOSURE

The present disclosure relates to methods of identifying and measuring the relative potency of multi-specific binding compounds, such as bispecific antibodies, that can activate reporter cells expressing a T cell receptor comprising gamma (γ) and delta (δ) chains.

### BACKGROUND

Over the last half century, enormous progress has been made in understanding the etiology and progression of cancer. However, while cancer-related death rates have fallen significantly over the last 20 years, cancer remains a significant source of morbidity and mortality worldwide. In 2020, cancer remained the second leading cause of death in the United States.

Numerous treatments for cancer have been proposed and tried over the last several decades. While early treatments focused on toxic chemicals that could kill cancer cells (i.e., chemotherapy), increased understanding in the field of immunology brought understanding that cells of the lymphoid cell lineage, and specifically T-lymphocytes (T cells), could search out, identify, and kill cancer cells. Consequently, much work has been done to develop cancer therapies based on T cells (e.g., CAR-T), that are able to more precisely target cancer cells, and eliminate them while reducing damage to surrounding, non-cancerous tissue.

Recently, excitement has grown around a type of T cell know as a γδT cell. While most T cells have a T cell receptor (TCR) comprising an alpha (α) chain and a beta (β) chain (αβT cells), γδT cells are characterized by expression of a TCR comprising a γ chain and a δ chain (γδTCR). Since their discovery in 1987, it has become apparent that γδT cells are functionally different from the more abundant αβT cells. For example, γδT cells are relatively rare in lymphoid organs and, instead, predominate in epithelial tissue and are abundant in circulation. More significantly, γδT cells can recognize target antigens in an MHC-independent manner, and thus can recognize and respond to a broad range of antigens, including proteins and lipids. Finally, γδT cells display NK-cell like innate activities that include killing infected cells as well as microorganisms and malignant cells. Thus, γδT cells hold great promise in immunotherapeutic applications such as cancer, infectious disease, and other immunity-related diseases.

There is a need in the art for assays for the selection of potent compounds that bind and activate γδT cells.

### SUMMARY

It has been shown that T cells can be physically recruited and linked to tumor surface antigens, thereby eliciting an antitumor response, using bispecific antibodies. Multispecific antibodies are engineered antibodies having at least two different antigen binding sites, whereby each antigen binding site specifically binds a unique epitope. T cell dependent bispecific antibodies (TDbAbs) are an example of a multispecific antibody in which one antigen binding site specifically binds a T cell signaling molecule (e.g., a TCR), and the other antigen binding site binds a target antigen on a target cell (e.g., tumor cell). Simultaneous binding of the TDbAb to the target antigen on the target cell and to a T cell signaling molecule elicits T cell recruitment to the target cell, which results in T cell activation and subsequent target cell depletion. In clinical use, the selection of TDbAbs must balance effectivity and safety. Weakly interacting TDbAbs may not have the desired therapeutic benefit, while TDbAbs that interact too strongly with the T cell signaling molecule may produce unwanted side effects, such as cytokine release syndrome (CRS). Furthermore, it is important that TDbAb compositions lack non-specific T cell activation. Thus, the development of therapeutic TDbAbs requires assays that accurately measure the potency of potential TDbAbs, to identify those best suited for further development. Such assays may also be used in the production process as a batch release assay. For example, such an assay may be used to determine if a manufactured batch of TDbAb meets a predefined potency criterion, such that the manufactured batch can be released for clinical use (e.g., administration to a patient).

An optimal assay for TDbAbs potency should be specific/selective, accurate, easy to use, and provide easy to understand output. The present disclosure provides an easy to use, accurate and scalable assay, that allows for detection of γδT cell activation by TDbAbs.

Provided herein is a method of detecting γδ T cell receptor (γδTCR)-mediated reporter cell activation by a γδTCR-dependent multispecific binding compound (γδ-TDMBC), wherein the γδ-TDMBC comprises a target antigen binding portion and a γδ TCR binding portion, the method comprising: a) contacting the γδ-TDMBC with a population of cells comprising: i) a reporter cell that expresses a γδ TCR, and that comprises a reporter gene responsive to activation of the reporter cell; and, ii) the target antigen; and, b) detecting expression of the reporter gene, wherein expression of the reporter gene indicates γδ TCR-mediated activation of the reporter cell.

One aspect is a method of determining the relative potency of a γδ-TDMBC comprising a γδ-TCR binding portion and a target antigen binding portion, the method comprising: a) contacting a known concentration of the γδ-TDMBC with a population of cells comprising: i) a reporter cell that expresses a γδ TCR, and that comprises a reporter gene responsive to activation of the reporter cell; and, ii) the target antigen; and, b) comparing the resulting level of reporter gene expression in a), with the level of reporter gene expression resulting from contacting the reporter cell of i) and the target antigen of ii) with the known concentration of a reference γδ TCR-dependent compound that binds the target antigen and the γδ TCR, thereby obtaining a measure of the relative potency of the γδ-TDMBC. In some aspects, step b) may comprise correlating the expression of the reporter gene as a function of γδ-TDMBC with a standard curve produced by contacting the population of T cells and the antigen with different concentrations of a reference γδ-TDMBC.

One aspect is a method of detecting the presence of a γδ-TDMBC comprising a γδ-TCR binding portion and a target antigen binding portion in a composition, comprising contacting the composition with a population of cells comprising: a) a reporter cell that expresses a γδ TCR, and that comprises a reporter gene responsive to activation of the reporter cell; and, b) the target antigen, wherein expression of the reporter gene indicates the presence of a γδ-TDMBC in the composition.

One aspect is a method of quantifying a γδ-TDMBC comprising a γδ-TCR binding portion and a target antigen binding portion, comprising: a) contacting the γδ-TDMBC with a population of cells comprising: i) a reporter cell that expresses a γδ TCR, and that comprises a reporter gene responsive to activation of the reporter cell; and, ii) the target antigen; and, b) correlating the level of expression of the reporter gene as a function of the γδ-TDMBC concentration with a standard curve produced by contacting a population of reporter cells and the target antigen with different known concentrations of the γδ-TDMBC, thereby quantifying the γδ-TDMBC.

One aspect is a method of determining the specificity of reporter cell activation by a γδ-TDMBC comprising a target antigen binding portion and a γδ TCR binding portion, comprising: i) contacting the γδ-TDMBC with a population of cells comprising a) a reporter cell that expresses a γδ TCR, and that comprises a reporter gene responsive to activation of the reporter cell; and, b) the target antigen; and, ii) contacting the γδ-TDMBC with a population of cells comprising a) a reporter cell of i) in the absence the antigen; and, comparing the expression of the reporter gene in i) with the expression of the reporter gene in ii); wherein the ratio of expression of the reporter gene in i) to the expression of the reporter gene in ii), is indicative of the specificity of the γδ-TDMBC for the target antigen.

In these methods, the reporter cell may be a reporter T cell, which may be CD3+. The γδ TCR expressed by the reporter cell may comprise a γ9 chain and/or a δ2 chain and may be a γ9δ2 TCR. The reporter cell may comprise one or more exogenous nucleic acid molecules encoding the TCR γ chain and/or δ chains, and these exogenous nucleic acid molecules may be stably integrated into the genome of the reporter cell. The reporter gene may comprise a nucleic acid molecule that comprises a nucleotide sequence encoding a reporter protein operably linked to a promoter that is responsive to activation of the reporter cell. The promoter may be responsive to T cell activation and may be selected from the group consisting of an NFAT promoter, an AP-1 promoter, an NFKB promoter, a FOXO promoter, a STAT3 promoter, a STAT5 promoter, and an IRF promoter. The reporter gene may comprise one or more response elements operably linked to the promoter.

In these methods, the reporter gene may comprise a nucleic acid molecule comprising a nucleotide sequence encoding a reporter protein operably linked to a promoter that is responsive to activation of the reporter cell, and one or more response elements operably linked to the promoter. The promoter may be a minimal promoter, which may be selected from the group consisting of a TK minimal promoter, a CMV minimal promoter, an SV40 minimal promoter, and a 1EF 1a minimal promoter. The reporter gene may encode a reporter protein, which may be any protein that is detectable, and which may be selected from a fluorescent protein, a luminescent protein, a chemiluminescent protein, and an enzyme.

In these methods, the target antigen may be immobilized on a physical structure, such as a plate or a bead, or it may be expressed by a target cell, and optionally on the surface of the target cell, present in the population of cells. In such methods, the ratio reporter cells to target cells may be about 1:1, about 1:2, about 1:3, about 1:4, about 1:5, about 1:6, about 1:7, about 1:8, about 1:9, about 1:10, about 1:20, about 1:50, or about 1:100. The target antigen may be a cancer-associated or a tumor-associated antigen and may be selected from the group consisting of EGFR, PSMA, CD1d, CD40, Nectin-4, and CD123. The γδ TCR binding portion of the γδ-TDMBC may specifically bind the γ chain of the TCR, which may be a γ9 chain. The γδ TCR binding portion of the γδ-TDMBC may specifically bind the δ chain of the TCR, which may be a δ2 chain. The γδ-TDMBC may bind EGFR, PSMA, CD1d, CD40, Nectin-4, and CD123. In these methods, the γδ-TDMBC may be a bispecific antibody.

In these methods, the γδ-TDMBC may be a γδ-TDbAb comprising a first single-domain antibody that specifically binds an epitope in a γδ TCR, wherein the first single-domain antibody may comprise CDR1, comprising or consisting of SEQ ID NO:5 in which X₁ is G or S, CDR2, comprising or consisting of SEQ ID NO:2, and/or CDR3, comprising or consisting of SEQ ID NO:14 in which X₂ can be any amino acid and X₃ is not R. The first single-domain antibody may comprise, or consist of, SEQ ID NO:16, wherein X₁ is G or S and wherein X₂ can be any amino acid and X₃ is not R. The γδ-TDbAbs may be a γδ-TDbAb comprising a second single-domain antibody that specifically binds a target antigen, wherein the second single-domain antibody may comprise CDR1, comprising or consisting of SEQ ID NO:45, CDR2, comprising or consisting of SEQ ID NO:46, and/or CDR3, comprising or consisting of SEQ ID NO:47. In some aspects, the second single-domain antibody may comprise, or consist of, SEQ ID NO:48.

The second single-domain antibody may comprise CDR1, comprising or consisting of SEQ ID NO:33, CDR2, comprising or consisting of SEQ ID NO:34, and/or CDR3, comprising or consisting of SEQ ID NO:35. The second single-domain antibody may comprise CDR1, comprising or consisting of SEQ ID NO:49, CDR2, comprising or consisting of SEQ ID NO:50, and/or CDR3, comprising or consisting of SEQ ID NO:51. The second single-domain antibody may comprise CDR1, comprising or consisting of SEQ ID NO:41, CDR2, comprising or consisting of SEQ ID NO:42, and/or CDR3, comprising or consisting of SEQ ID NO:43. The second single-domain antibody may comprise CDR1, comprising or consisting of SEQ ID NO:37 in which X₄ is G or S, CDR2, comprising or consisting of SEQ ID NO:38 in which X₅ is A or T, and/or CDR3, comprising or consisting of SEQ ID NO:39 in which X₆ is Y or F. The second single-domain antibody may comprise, or consist of, SEQ ID NO:48, SEQ ID NO:36, SEQ ID NO:52, SEQ ID NO:44, SEQ ID NO:40, SEQ ID NO: 56, SEQ ID NO: 60, SEQ ID NO: 64, SEQ ID NO: 68, SEQ ID NO: 72, SEQ ID NO: 76, or SEQ ID NO: 80.

The reporter cell may not express a functional TCR α and/or β chain and may comprise one or more knockout mutations within a TCR α chain locus and/or TCR β chain locus, or within genetic elements controlling expression of either or both loci. In these methods, the reporter cell may not express EGFR, PSMA, CD1d, CD40, CD123, and Nectin-4.

One aspect is a reporter cell expressing a γ9δ2 T cell receptor (γδTCR) and comprising a reporter gene responsive to activation of the reporter cell. In some aspects, the reporter cell may be a reporter T cell, which may be CD3+, and/or which may be a Jurkat cell or a CTLL-2 cell. In some aspects, the reporter cell may comprise one or more exogenous nucleic acid molecules encoding the TCR γ chain and/or δ chains, and these exogenous nucleic acid molecules may be stably integrated into the genome of the reporter cell. In some aspects, the reporter gene may comprise a nucleic acid molecule that comprises a nucleotide sequence encoding a reporter protein operably linked to a promoter that is responsive to activation of the reporter cell. The promoter may be responsive to T cell activation and may be selected from the group consisting of an NFAT promoter, an AP-1 promoter, an NFκB promoter, a FOXO promoter, a STAT3 promoter, a STAT5 promoter, and an IRF promoter. The reporter gene may comprise one or more response elements operably linked to the promoter. In some aspects, the reporter gene may comprise a nucleic acid molecule comprising a nucleotide sequence encoding a reporter protein operably linked to a promoter that is responsive to activation of the reporter cell, and one or more reporter cell activation response elements operably linked to the promoter. The promoter may be a minimal promoter, which may be selected from the group consisting of a TK minimal promoter, a CMV minimal promoter, an SV40 minimal promoter, and a 1EF1α minimal promoter. The reporter cell activation response elements may be T cell activation response elements, which may be selected from the group consisting of an NFAT gene response element, an AP-1 gene response element, an NFκB gene response element, a FOXO gene response element, a STAT3 gene response element, a STAT5 gene response element, and an IRF gene response element. In some aspects, the reporter gene may comprise at least two reporter cell activation response elements, which may be arranged as tandem repeats. The reporter cell activation response element(s), or tandem repeats thereof, may be positioned 5′ of the nucleic acid sequence encoding the reporter protein, and may be positioned 5′ of the promoter. In some aspects, the reporter gene may encode a reporter protein, which may be any protein that is detectable, and which may be selected from a fluorescent protein, a luminescent protein, a chemiluminescent protein, and an enzyme. In some aspects, the reporter cell may not express a functional TCR α and/or β chain, and may comprise one or more knockout mutations within a TCR α chain locus and/or a TCR β chain locus, or within genetic elements controlling expression of either or both loci. In some aspects, the reporter cell does not express EGFR, PSMA, CD1d, CD40, Nectin-4, and CD123.

One aspect is a kit comprising a reporter cell of the disclosure, and the kit may comprise a pharmaceutically acceptable excipient.

### BRIEF DESCRIPTION OF THE DRAWINGS

FIG. 1A-FIG. 1B illustrate an exemplary T cell activation bioassay for a multispecific binding compound. FIG. 1A. illustrates an embodiment of the assay of the disclosure. The assay comprises a reporter cell expressing a γ9δ2TCR, and a reporter gene (luciferase), the expression of which is responsive to activation of the reporter T cell. The illustrated assay also comprises a target cell expressing a target antigen. FIG. 1B illustrates addition of a TDbAb to the assay. The TDbAb binds the TCR and to the target antigen causing activation of the reporter cell, which results in production of luciferase and subsequent luminescence.

FIG. 2 shows the stability of a bispecific binding compound, assessed using estimated relative potency, when stored at 5° C. for various periods of time, using an assay of the disclosure.

FIG. 3 shows the stability of a bispecific binding compound, assessed using estimated relative potency, when stored at 20° C. for various periods of time, using an assay of the disclosure.

FIG. 4 shows the stability of a bispecific binding compound, assessed using estimated relative potency, when stored at 40° C. for various periods of time, using an assay of the disclosure.

FIG. 5A-FIG. 5B show detection of immobilized CD1d (FIG. 5A) or immobilized PSMA (FIG. 5B) using a TDbAb and reporter cells of the disclosure.

FIG. 6 shows the presence of a background signal when the γδ-TDMBC is incubated with the reporter cell line.

FIG. 7 shows knock-out of the target antigen from the reporter cell line.

FIG. 8 shows luciferase signals in the reporter cell line with, and without, target antigen knock-out.

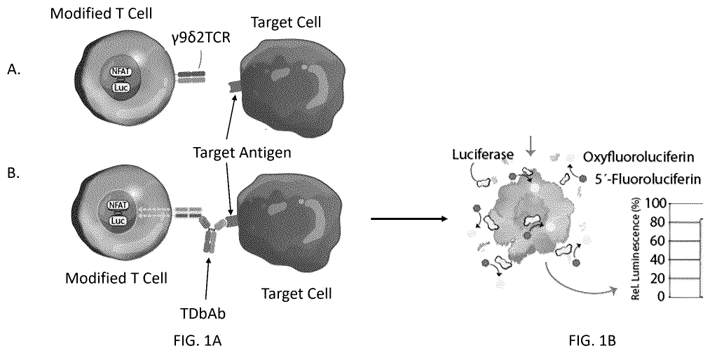

### DETAILED DESCRIPTION

The disclosure provides a method for detecting γδ TCR-mediated activation of reporter cells. More specifically, the present disclosure provides assays for measuring the ability of a γδ T cell receptor (γδTCR)-dependent, multispecific binding compound (γδ-TDMBC), such as a TDbAb, to activate a γδ-expressing reporter cell in the presence of a target antigen recognized by the γδ-TDMBC. Such assays may be used to measure the relative potency of the γδ-TDMBC. Reporter cells used in the disclosed assays express a γδ TCR, and have been modified to include a reporter gene such that activation of the γδ-expressing reporter cell results in production of a detectable reporter protein, allowing for rapid and easy detection of the activated reporter cell. Thus, a method of the disclosure may generally be practiced by contacting a γδ-TDMBC with a target antigen and a population of reporter cells comprising a reporter gene that is responsive to reporter cell activation, and assaying the reporter cells for expression of the reporter gene, wherein expression of the reporter gene indicates γδ-TDMBC-induced activation of the reporter cell. The expression level of the reporter gene may be compared to the expression level obtained by contacting a reference γδ-TDMBC with the target antigen and a reporter cell, thereby determining the relative potency of the first γδ-TDMBC.

Before the present disclosure is further described, it is to be understood that the disclosure is not limited to particular embodiments described, as such may, of course, vary. It is also to be understood that the terminology used herein is for the purpose of describing particular embodiments only, and is not intended to be limiting, since the scope of the present disclosure will be limited only by the claims.

It must be noted that as used herein and in the appended claims, the singular forms “a,” “an,” and “the” include plural referents unless the context clearly dictates otherwise. For example, a compound refers to one or more compound molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Publications disclosed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed. All publications mentioned herein are incorporated herein by reference to disclose and describe the methods and/or materials in connection with which the publications are cited.

A γδ-TDMBC (γδ-TCR dependent multispecific binding compound) is a compound having two or more binding sites, each of which binds a unique epitope, wherein one of the binding sites specifically binds an epitope in a γδ TCR. In some aspects a second binding site of the γδ-TDMBC may bind a target antigen. One example of a TDMBC is a multi-specific antibody, such as a bispecific antibody. Multi-specific antibodies are engineered antibodies having at least two different antigen binding sites, whereby each antigen binding site specifically binds a unique epitope. γδ T cell dependent bispecific antibodies (γδ-TDbAbs), disclosed herein, are bispecific antibodies in which one antigen binding site specifically binds a γδ TCR and the second antigen binding site specifically binds to a target antigen. In some aspects, the γδ-TDMBC may be a multi-specific antibody comprising at least two single domain antibodies, wherein at least one single domain antibody specifically binds to a γδ TCR, and preferably to the V chain (e.g., Vδ2 chain), and at last one other single domain antibody specifically binds to the target antigen (e.g., CD1d, CD40, CD123, PSMA, Nectin-4 and EGFR). In some aspects, the γδ-TDMBC may be a bispecific antibody comprising two single domain antibodies, wherein one single domain antibody specifically binds to a γδ TCR, and preferably to the Vδ chain (e.g., Vδ2 chain), and the other single domain antibody specifically binds to the target antigen (e.g., CD1d, CD40, CD123, PSMA, Nectin-4 and EGFR). As used herein, “specifically binds” refers to differential binding of an antigen binding site to at least two different epitopes. “Specifically binds” means that an antigen binding site binds a target molecule with an affinity, or avidity, significantly greater than its affinity, or avidity, for a molecule unrelated to the target molecule. For example, an antigen binding site that specifically binds to a TCR γ chain protein means that the affinity, or avidity, of the antigen binding site for the TCR γ chain protein is significantly greater than its affinity, or avidity, for a protein unrelated to the TCR γ chain protein, such as a TCR α chain. In some aspects of the disclosure, an antigen binding site of a TDMBC binds an epitope with a KD of about 1 μM or less, 0.1 μM or less, 0.01 μM or less, or about 1 nM or less. In some aspects, the γδ-TDMBC specifically binds the γ chain of a TCR. In some aspects, the γδ-TDMBC specifically binds the δ chain of a TCR. In some aspects, the γδ-TDMBC specifically binds a γ9 chain of a TCR. In some aspects, the γδ-TDMBC specifically binds a δ2 chain of a TCR.

As used herein, “target antigen”, “antigen target”, “antigen”, and the like, refer to any biomolecule, such as a protein, glycoprotein, lipoprotein, or sugar, that may be expressed by a cell against which it is desirable to elicit a T cell response. In methods of the disclosure, target antigens may, but need not be expressed by target cells, such as on the surface of a target cell. In some aspects, the antigen may be immobilized to a surface, such as a plate (e.g., a microtiter plate), or a bead (e.g., a latex bead). Useful target antigens may be found, for example, on the surfaces of tumor cells, on the surfaces of virus-infected cells, on the surfaces of bacteria-infected cells, and on the surfaces of other diseased cells. Target antigens may be biomolecules (e.g., proteins) from any vertebrate source, including mammals such as primates (e.g., humans) and rodents (e.g., mice and rats), or from viral or bacterial sources. Where reference is made to a specific target antigen herein, the term encompasses the “full-length”, unprocessed target antigen as well as any form of the target antigen that results from processing in the target cell. The term also encompasses naturally occurring variants of the target antigen, e.g., splice variants or allelic variants. Exemplary human target proteins useful as antigens include, but are not limited to, CD1d, epidermal growth factor receptor (EGFR), prostate-specific membrane antigen (PSMA), cluster of differentiation (CD) 40 (CD40), Nectin-4, and CD123.

A “reporter cell” refers to a cell that expresses a γδ TCR, and that has been genetically modified by the hand of man to possess a reporter gene that is responsive to γδ TCR-mediated activation of the reporter cell. Reporter cells of the disclosure may express the γδ TCR naturally or they may express the γδ TCR as a result of genetic manipulation of the cell by the hand of man (e.g., introduction into the cell of exogenous nucleic acid molecules encoding γ and δ TCR chains). “Activation of the reporter cell”, “reporter cell activation”, and the like, refer to a change in the physiological state and/or composition of the reporter cell upon binding of, at least, the γδ TCR by the γδ-TDMBC. Reporter cell activation involves a γδ TCR-mediated signal cascade and comprises increased, decreased, and/or new, production and/or activation of cellular proteins and other molecules, that can bind control elements (e.g., promoters, enhancers, operators, etc.) within the genome, thereby regulating (e.g., activating) gene expression. Thus, reporter cell activation results in one or more cellular responses, examples of which include, but are not limited to, altered gene expression, cell proliferation, cell differentiation, cytokine production and/or secretion, altered qualitative or quantitative composition of cell surface proteins, cytotoxic effector molecule production and/or release, and cytotoxic activity. In some aspects of the disclosure, a reporter cell may be produced by genetically modifying a T cell. In such aspects, the reporter cell may be referred to as a reporter T cell.

“T cell,” refers to a type of blood cell known as a T-lymphocyte that matures in the thymus, and is distinguished from other lymphocytes, such as B cells, by, at least, the presence of a T cell receptor on the cell surface. T cells useful for producing reporter cells of the disclosure include any cell expressing a functional TCR and possessing a TCR-activated signaling cascade that regulates transcription. Examples of such cells include, but are not limited to, T-helper cells (CD4⁺ cells), cytotoxic T cells (CD8⁺ cells), natural killer T cells, T-regulatory cells (Treg) and γδT cells. T cells used in aspects of the instant disclosure may either be isolated from humans or animals, obtained from culture, or obtained from a commercially available source. Non-limiting examples of commercially available T cell lines include lines BCL2 (AAA) Jurkat (ATCC® CRL-290™), BCL2 (S70A) Jurkat (ATCC® CRL-290™), BCL2 (S87A) Jurkat (ATCC® CRL-290™), BCL2 Jurkat (ATCC® CRL-2899™), Neo Jurkat (ATCC® CRL-2898™), TALL-104 cytotoxic human T cell line (ATCC® #CRL-11386), and CTLL-2 (ATCC® TIB-214™). Further examples include but are not limited to mature T cell lines, e.g., such as Deglis, EBT-8, HPB-MLp-W, HUT 78, HUT 102, Karpas 384, Ki 225, My-La, Se-Ax, SKW-3, SMZ-1 and T34; and immature T cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Jchikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC® TIB-153), J45.01 (ATCC® CRL-1990), J.CaM1.6 (ATCC® CRL-2063), RS4; 11 (ATCC® CRL-1873), CCRF-CEM (ATCC® CRM-CCL-119); and cutaneous T cell lymphoma lines, e.g., HuT78 (ATCC® CRM-TIB-161), MJ[G11] (ATCC® CRL-8294), HuT102 (ATCC® TIB-162). In some aspects, a reporter T cell of the disclosure may be CD4⁺ (i.e., expresses the CD4 protein), CD3+ and/or CD8⁺. In some aspects, the reporter T cell of the disclosure may be a CD4⁻ (i.e., does not express the CD4 protein), and/or CD8⁻ T cell. In some aspects, a reporter T cell of the disclosure may be produced from an immortalized T cell (e.g., a T cell line). In some aspects, a reporter T cell of the disclosure may be produced from a Jurkat cell. In some aspects, a reporter T cell of the disclosure may be produced from a CTLL-2 T cell.

As used herein, “T cell receptor” refers to T cell receptors as generally understood in the field of immunology. TCRs are heterodimers composed of two different peptide chains: an α chain and a β chain, or a γ chain and a δ chain. Reporter cells of the disclosure express a TCR receptor comprising a γ chain and a δ chain. The TCR-gamma gene locus is known to comprise at least 12 functional variable (V) gene segments, each encoding a γ chain variable region, while the TCR-delta locus is known to comprise at least 8 V gene segments, each encoding a δ chain variable region. In some aspects, reporter cells of the disclosure may express a γδTCR comprising a V region from any Vγ gene segment. In some aspects, reporter cells of the disclosure may express a γδTCR comprising a V region from any Vδ gene segment. In some aspects, reporter cells of the disclosure may express a γδTCR comprising a V region from a Vγ9 gene segment. In some aspects, reporter cells of the disclosure may express a γδTCR comprising a V region from a Vδ2 gene segment. In some aspects, a reporter cell of the disclosure comprises a TCR comprising Vγ9 and Vδ2 regions (a Vγ9Vδ2 TCR).

As used herein, “reporter gene” refers to a nucleic acid molecule comprising a nucleotide sequence encoding a reporter protein, the presence or activity of which can be detected or measured, operably linked to a promoter and, optionally, to an activated reporter cell response element. The phrase “reporter gene”, as used herein, does not necessarily indicate the presence of genetic elements such as exons, intron, splicing signals, and the like. While in some aspects such elements may be present, a reporter gene of the disclosure may comprise an open reading frame that lacks introns and exons. For example, a reporter gene of the disclosure may comprise a single open reading frame encoding a fluorescent protein operably linked to a promoter and, optionally, to an activated reporter cell response element. In some aspects, the reporter protein may, under appropriate conditions, produce a detectable signal that allows detection for indicating the presence and/or quantity of the reporter protein. Examples of suitable reporter proteins include, but are not limited to, fluorescent molecules, such as fluorescent proteins, luminescent molecules, such as luminescent proteins, chemiluminescent molecules, such as chemiluminescent proteins, and enzymes, such as alkaline phosphatase or beta-galactosidase. One example of a luminescent protein is luciferase. Luciferases are a class of luminescent proteins that are derived from many sources and include firefly luciferase (from the species, Photinus pyralis), Renilla luciferase from sea pansy (Renilla reniformis), click beetle luciferase (from Pyrearinus termitilluminans), marine copepod Gaussia luciferase (from Gaussia princeps), and deep sea shrimp Nano luciferase (from Oplophorus gracilirostris). Firefly luciferase catalyzes the oxygenation of luciferin to oxyluciferin, resulting in the emission of a photon of light, while other luciferases, such as Renilla, emit light by catalyzing the oxygenation of coelenterazine. The wavelength of light emitted by different luciferase forms and variants can be read using different filter systems, which facilitates multiplexing. The amount of luminescence is proportional to the amount of luciferase expressed in the cell. In some aspects, the reporter gene may be multi-cistronic, meaning that it encodes more than one reporter protein, which may be a fusion protein. The use of a multi-cistronic reporter gene allows the use of duel reporter proteins (e.g., fluorescent proteins having two different colors), and/or allows the use of destabilizing sequences (e.g., PEST sequence), that may allow reduced half-life of the fusion protein, thereby reducing “leaky” expression. In some aspects, the reporter protein may be an intracellular protein (i.e., it remains within the cell). In some aspects, the reporter protein may be a secreted protein.

“Responsive to reporter cell activation”, “responsive to activation of the reporter cell”, and the like, are phrases used in reference to genetic elements (e.g., genes, control elements such as promoters, response elements, etc.) and refer to the fact that the state or activity of the referenced element is altered by activation of the reporter cell. For example, a gene that is responsive to reporter cell activation means the gene is expressed (i.e., transcribed) when the reporter cell is in an activated state. Similarly, a promoter that is responsive to reporter cell activation is a promoter that becomes active (e.g., promotes transcription of a gene to which it is operably linked) when the reporter cell is in an activated state. Likewise, an activated reporter cell response element is a response element that affects promoter/transcription activity (e.g., enhances transcription) when the reporter cell is activated.

“Operably linked” refers to the relative positioning, with or without intervening sequence such as a spacer or linker sequence, of two or more nucleotide sequences, so that they are in a relationship wherein an event (i.e., binding of a molecule) at one or more nucleotide sequences causes an effect at one or more different nucleotide sequences. For example, a promoter that is operably linked to a coding sequence, such as an open reading frame, can drive expression of the coding sequence. Such a coding sequence may also be referred to as “being under control of”, or “controlled by”, the promoter. As a further example, a response element operably linked to a coding sequence, or a promoter driving expression of the coding sequence, will allow or enhance expression of the linked coding sequence.

“Response element” refers to a cis-acting DNA sequence that confers responsiveness on a gene, such responsiveness being mediated though interaction with the DNA-binding domain of a cellular molecule such as a transcription factor. An “activated reporter cell response element”, “activated cell response element”, “activation response element”, and the like, refer to a response element that affects promoter/transcription activity (e.g., enhances transcription) when the reporter cell is activated. One example of a response element is an enhancer. In the present disclosure, operably linking a response element to a reporter gene may enhance activity of an operably linked promoter when the reporter cell is in an activated state. Thus, suitable response elements useful for practicing aspects of the instant disclosure includes any response element that when operably linked to the reporter gene makes expression of the reporter gene responsive to activation of the reporter cell. In some aspects, a response element may comprise a T cell activation response element. Examples of such response elements include, but are not limited to, an NFAT gene response element, an AP-1 gene response element, an NFκB gene response element, a FOXO gene response element, a STAT3 gene response element, a STAT5 gene response element, and an IRF gene response element. Response elements may be arranged as tandem repeats (such as about any of 2, 3, 4, 5, 6, 7, 8, or more tandem repeats), which may increase the responsiveness of an operably linked promoter or gene. A response element(s) may be positioned 5′ or 3′ to the reporter gene. A response element(s) may be located at a site 5′ from the promoter.

One aspect of the disclosure is a method of detecting γδ TCR-mediated reporter cell activation by a γδ-TDMBC having a γδ TCR binding site and a target antigen binding site, the method comprising:
a) contacting the γδ-TDMBC with a population of cells comprising: i) a reporter cell of the disclosure that expresses a γδ TCR, and that comprises a reporter gene responsive to activation of the reporter cell; and, ii) the target antigen; and,
b) detecting expression of the reporter gene, wherein expression of the reporter gene indicates γδ TCR-mediated activation of the reporter cell. In some aspects, detecting expression of the reporter gene comprises detecting a reporter protein encoded by the reporter gene.

One aspect of the disclosure is a method of determining the relative potency of a γδ-TDMBC having a γδ TCR binding site and a target antigen binding site, the method comprising: a) contacting a known concentration of the γδ-TDMBC with a population of cells comprising: i) a reporter cell of the disclosure that expresses a γδ TCR, and that comprises a reporter gene responsive to activation of the reporter cell; and, ii) the target antigen; and, b) comparing the resulting level of reporter gene expression with the level of reporter gene expression resulting from contacting a reporter cell and the target antigen with the known concentration of a reference γδ-TDMBC that binds the target antigen and the γδ TCR, thereby obtaining a measure of the relative potency of the first γδ-TDMBC. In some aspects, the method comprises detecting expression of the reporter gene by detecting a reporter protein encoded by the reporter gene. In some aspects, step b) comprises correlating the expression of the reporter gene as a function of γδ-TDMBC concentration with a standard curve produced by contacting the population of T cells and the antigen with different concentrations of the reference γδ-TDMBC.

One aspect of the disclosure is a method of detecting the presence of a γδ-TDMBC comprising a γδ TCR binding site and a target antigen binding site in a composition, the method comprising contacting the composition with a population of cells comprising: a) a reporter cell of the disclosure that expresses a γδ TCR, and that comprises a reporter gene responsive to activation of the reporter cell; and, b) the target antigen, wherein expression of the reporter gene indicates the presence of a γδ-TDMBC in the composition. In some aspects, the method comprises detecting expression of the reporter gene by detecting a reporter protein encoded by the reporter gene.

One aspect of the disclosure is a method of quantifying a γδ-TDMBC comprising a γδ TCR binding site and a target antigen binding site, the method comprising: a) contacting the γδ-TDMBC with a population of cells comprising: i) a reporter cell of the disclosure that expresses a γδ TCR, and that comprises a reporter gene responsive to activation of the reporter cell; and, ii) the target antigen; and, b) correlating the expression of the reporter gene as a function of the γδ-TDMBC concentration with a standard curve produced by contacting the population of reporter cells and target antigen with different concentrations of the γδ-TDMBC, thereby quantifying the γδ-TDMBC. In some aspects, the method comprises detecting expression of the reporter gene by detecting a reporter protein encoded by the reporter gene. In some aspects, step b) comprises correlating the expression of the reporter gene as a function of γδ-TDMBC concentration with a standard curve produced by contacting the population of T cells and the antigen with different concentrations of the reference γδ-TDMBC.

One aspect of the disclosure is a method of determining the specificity of reporter cell activation by a γδ-TDMBC comprising a target antigen binding site and a γδ TCR binding site, the method comprising:
i) contacting the γδ-TDMBC with a population of cells comprising a) a reporter cell of the disclosure that expresses a γδ TCR, and that comprises a reporter gene responsive to activation of the reporter cell; and, b) the target antigen; and,
ii) comparing the expression of the reporter gene in i) with the expression of the reporter gene in a reporter cell, when the reporter cell is contacted with the γδ-TDMBC in the absence of the antigen; wherein the ratio of expression of the reporter gene in i) to the expression of the reporter gene in ii), is indicative of the specificity of the γδ-TDMBC for the target antigen. In some aspects, the method comprises detecting expression of the reporter gene by detecting a reporter protein encoded by the reporter gene.

In these methods, the reporter cell may be a reporter T cell. In these methods, the reporter T cell may be produced by modification of a Jurkat cell or a CTLL-2 T cell. In these methods, the reporter T cell may comprise one or more knock out mutations within a TCR α chain locus, or a TCR β chain locus, or within genetic elements controlling expression of either or both loci. In these methods, the reporter cell may lack a functional TCR α chain gene and/or TCR β chain gene. In these methods, the reporter cell may lack a functional αβTCR.

In these methods, the γδ TCR expressed by the reporter cell may comprise a γ9 chain. In these methods, the γδ TCR expressed by the reporter cell may comprise δ2 chain. In these methods, the γδ TCR expressed by the reporter cell may comprise a γ9δ2 TCR. In these methods, the γδ TCR may comprise a γ9δ2 TCR and the γδ-TCR binding site may specifically bind the γ9 chain of the TCR. In these methods, the γδ TCR may comprise a γ9δ2 TCR and the γδ-TCR binding site may specifically bind the δ2 chain of the γδ TCR. In these methods, the reporter cell may comprise one or more exogenous nucleic acid molecules encoding the γ and δ chains of the TCR. In these methods, the one or more exogenous nucleic acid molecules may be stably integrated into the genome of the reporter cell.

In these methods, the target antigen may be a cancer-associated or tumor-associated antigen. In these methods, the target antigen may be selected from the group consisting of CD1d, CD40, CD123, PSMA, Nectin-4, and EGFR. In these methods, the target antigen may be immobilized on a physical structure, such as, a bead, a tube, or a microtiter plate or aggregated by multimerization and crosslinking techniques (e.g. chemical or by multivalent proteins). In these methods, the population of cells may comprise a target cell expressing the target antigen. In these methods, the target antigen may be displayed on the surface of the target cell.

In these methods, the target antigen binding site may specifically bind a cancer-associated or tumor-associated antigen. In these methods, the target antigen binding site may specifically bind a target antigen selected from the group consisting of CD1d, CD40, CD123, PSMA, Nectin-4, and EGFR. In these methods, the γδ-TDMBC may comprise a TDbAb.

In these methods, the reporter gene may comprise a nucleic acid molecule comprising a nucleotide sequence encoding a reporter protein, operably linked to a promoter responsive to reporter cell activation, and, optionally, to one or more additional response elements. In these methods, the promoter may be selected from any promoter known to be responsive to activation of the reporter cell. For example, in aspects in which the reporter cell is a reporter T cell, the promoter may be any promoter that is responsive to T cell activation. In these methods, the promoter may be selected from the group consisting of a nuclear factor of activated T-cells (NFAT) gene promoter, an interleukin-2 (IL-2) gene promoter, an activator protein 1(AP-1) gene promoter, a nuclear factor kappa B subunit (NFκB) gene promoter, a Forkhead Box subfamily 0 (FOXO) gene promoter, a Signal Transducer and Activator of Transcription 3 (STAT3) gene promoter, a Signal Transducer and Activator of Transcription 5 (STATS) gene promoter, and an interferon regulatory factor (IRF) gene promoter.

In these methods, the reporter gene may comprise a nucleic acid molecule comprising a nucleotide sequence encoding a reporter protein under the control of a promoter and operably linked to one or more response elements responsive to reporter cell activation. In such methods, the promoter controlling the nucleotide sequence encoding the reporter protein may be a minimal promoter. “Minimal promoter” refers to the minimal nucleotide sequence from a promoter that is necessary for expression of a coding sequence under control of the promoter. Minimal promoters, which may be obtained commercially, are synthetic promoters that have been designed to provide minimal (i.e., no or very low level) transcription of a nucleic acid sequence operably linked thereto in the absence of a stimulatory signal such as from an enhancer. The use of minimal promoters is known in the art and is also disclosed in US2020/0182882 and U.S. Ser. No. 10/690,678, both of which are incorporated herein by reference in their entirety. In these methods, the minimal promoter may be a thymidine kinase (TK) minimal promoter, a cytomegalovirus (CMV) minimal promoter, a simian virus (SV) 40 minimal promoter, or an elongation factor (1EF1α) minimal promoter.

In these methods, the response element may be any response element that when operably linked to the reporter gene makes the reporter gene responsive to activation of the reporter cell. In these methods, the one or more response elements may comprise a T cell activation response element. In these methods, the one or more response elements may be selected from the group consisting of an NFAT gene response element, an AP-1 gene response element, an NFκB gene response element, a FOXO gene response element, a STAT3 gene response element, a STAT5 gene response element, or an IRF gene response element. In some aspects, response elements may be arranged as tandem repeats (such as about any of 2, 3, 4, 5, 6, 7, 8, or more tandem repeats). In these methods, the response element(s) may be positioned 5′ or 3′ to the reporter gene. In these methods, the response element(s) may be located at a site 5′ from the promoter. In these methods, the nucleic acid molecule comprising the reporter gene may be stably integrated into the genome of the reporter cell. In these methods, the reporter cell does not express the target antigen.

In these methods, the reporter gene may encode a fluorescent protein, a luminescent protein, a chemiluminescent protein, or an enzyme. In some aspects, the luminescent protein may be luciferase, which may be firefly luciferase, click beetle luciferase, marine copepod Gaussia luciferase, or deep-sea shrimp Nano luciferase.

Reporter cells of the disclosure may comprise any additional alterations that improve their suitability for use in assays of the disclosure. For example, in some methods of the disclosure it may be desirable if the reporter cell does not express the target antigen, or a protein that immunologically cross-reacts with the target protein. In these methods, reporter cells of the disclosure may be modified so that they do not express the target antigen, or a protein that cross-reacts with the target antigen. Thus, in these methods, a reporter cell of the disclosure may not express the target antigen or a molecule that immunologically cross-reacts with the target antigen. In these methods, a reporter cell of the disclosure may not express an antigen selected from the group consisting of CD1d, CD40, CD123, PSMA, Nectin-4, and EGFR.

In these methods, when the population of cells comprises a target cell expressing the target antigen, the ratio of reporter cells to target cells may be about 1:1, about 1:2, about 1:3, about 1:4, about 1:5, about 1:6, about 1:7, about 1:8, about 1:9, about 1:10, about 1:20, about 1:50, or about 1:100.

In these methods, the γδ-TDMBC may be at or within a concentration range of at least about 1 picomolar (pM), about 5, pM, about 25 pM, about 50 pM, about 100, pM, about 250 pM, about 500 pM, 750 pM, about 1,000 pM, about 1,500 pM, about 2,000 pM, about 2,500 pM, about 5,000, pM, about 10,000 pM, about 15,000 pM, or about 20,000 pM.

In these methods, reporter gene expression may be detected after more than about any of 5 minutes, 15 minutes, 10 minutes, 1 hr., 2 hr., 3 hr., 4 hr., 5 hr., 6 hr., 7 hr., 8 hr., 9 hr., 10 hr., 12 hr., 16 hr., 20 hr., or 24 hr. after contacting the cells with the T cell dependent bispecific binding molecule. In these methods, the reporter gene or molecule may be detected between any of about 5 and 15 minutes, 15 and 20 minutes, 30 minutes and 1 hr., 1 hr. and about 24 hr., about 1 hr. and about 12 hr., about 1 hr. and about 8 hr., about 1 hr. and about 6 hr., about 1 hr. and about 4 hr., about 1 hr. and about 2 hr., about 4 hr. and about 24 hr., about 4 hr. and about 12 hr., about 4 hr. and about 8 hr., about 8 hr. and about 24 hr., about 8 hr. and about 12 hr., about 16 hr. and about 24 hr., about 16 hr. and about 20 hr., or about 20 hr. and about 24 hr. after contacting the cells with the T cell dependent bispecific binding molecule.

In these methods, the γδ-TDbAbs may comprise a first single-domain antibody and a second single-domain antibody, wherein the first single-domain antibody specifically binds an epitope in a γδ TCR, and wherein the second single-domain antibody specifically binds a target antigen. In some aspects, the first single domain antibody specifically binds a TCR γ chain or a TCR δ chain. In some aspects, the first single domain antibody specifically binds a TCR δ chain. In some aspects, the first single domain antibody specifically binds a TCR δ2 chain In some aspects, the first single-domain antibody may comprise CDR1, comprising or consisting of SEQ ID NO:5 in which X₁ is G or S, CDR2, comprising or consisting of SEQ ID NO:2, and/or CDR3, comprising or consisting of SEQ ID NO:3 in which X₂ can be any amino acid and X₃ is not R, wherein the first single-domain antibody specifically binds a TCR δ chain, such as a δ2 chain. In some aspects, the first single-domain antibody may comprise, or consist of, an amino acid sequence at least 80% identical, at least 85% identical, at least 90% identical, at least 95% identical, at least 97% identical, or at least 99% identical to SEQ ID NO:16, wherein the amino acid sequence comprises CDR1, comprising or consisting of SEQ ID NO:5 in which X₁ is G or S, CDR2, comprising or consisting of SEQ ID NO:2, and CDR3, comprising or consisting of SEQ ID NO:3 in which X₂ can be any amino acid and X₃ is not R, and wherein the first single-domain antibody specifically binds a TCR δ chain, such as a δ2 chain. In some aspects, the first single-domain antibody may comprise, or consist of, SEQ ID NO:16, wherein X₁ is S or G, X₂ can be any amino acid, and X₃ is not R.

In some aspects, the second single-domain antibody may comprise CDR1, comprising or consisting of SEQ ID NO:45, CDR2, comprising or consisting of SEQ ID NO:46, and/or CDR3, comprising or consisting of SEQ ID NO:47, wherein the second single-domain antibody specifically binds CD1d. In some aspects, the second single-domain antibody may comprise, or consist of, an amino acid sequence at least 80% identical, at least 85% identical, at least 90% identical, at least 95% identical, at least 97% identical, or at least 99% identical to SEQ ID NO:48, wherein the amino acid sequence comprises CDR1, comprising or consisting of SEQ ID NO:45, CDR2, comprising or consisting of SEQ ID NO:46, and CDR3, comprising or consisting of SEQ ID NO:47, and wherein the second single-domain antibody specifically binds CD1d. In some aspects, the second single-domain antibody may comprise, or consist of, SEQ ID NO:48. In some aspects, the second single-domain antibody may comprise CDR1, comprising or consisting of SEQ ID NO:33, CDR2, comprising or consisting of SEQ ID NO:34, and/or CDR3, comprising or consisting of SEQ ID NO:35, wherein the second single-domain antibody specifically binds EGFR. In some aspects, the second single-domain antibody may comprise, or consist of, an amino acid sequence at least 80% identical, at least 85% identical, at least 90% identical, at least 95% identical, at least 97% identical, or at least 99% identical to SEQ ID NO:36, wherein the amino acid sequence comprises CDR1, comprising or consisting of SEQ ID NO:33, CDR2, comprising or consisting of SEQ ID NO:34, and CDR3, comprising or consisting of SEQ ID NO:35, and wherein the second single-domain antibody specifically binds EGFR. In some aspects, the second single-domain antibody may comprise, or consist of, SEQ ID NO:36. In some aspects, the second single-domain antibody may comprise CDR1, comprising or consisting of SEQ ID NO:49, CDR2, comprising or consisting of SEQ ID NO:50, and/or CDR3, comprising or consisting of SEQ ID NO:51, wherein the second single-domain antibody specifically binds CD40. In some aspects, the second single-domain antibody may comprise, or consist of, an amino acid sequence at least 80% identical, at least 85% identical, at least 90% identical, at least 95% identical, at least 97% identical, or at least 99% identical to SEQ ID NO:52, wherein the amino acid sequence comprises CDR1, comprising or consisting of SEQ ID NO:49, CDR2, comprising or consisting of SEQ ID NO:50, and CDR3, comprising or consisting of SEQ ID NO:51, and wherein the second single-domain antibody specifically binds CD40. In some aspects, the second single-domain antibody may comprise, or consist of, SEQ ID NO:52. In some aspects, the second single-domain antibody may comprise CDR1, comprising or consisting of SEQ ID NO:41, CDR2, comprising or consisting of SEQ ID NO:42, and/or CDR3, comprising or consisting of SEQ ID NO:43, wherein the second single-domain antibody specifically binds PSMA. In some aspects, the second single-domain antibody may comprise, or consist of, an amino acid sequence at least 80% identical, at least 85% identical, at least 90% identical, at least 95% identical, at least 97% identical, or at least 99% identical to SEQ ID NO:44, wherein the amino acid sequence comprises CDR1, comprising or consisting of SEQ ID NO:41, CDR2, comprising or consisting of SEQ ID NO:42, and CDR3, comprising or consisting of SEQ ID NO:43, and wherein the second single-domain antibody specifically binds PSMA. In some aspects, the second single-domain antibody may comprise, or consist of, SEQ ID NO:44. In some aspects, the second single-domain antibody may comprise CDR1, comprising or consisting of SEQ ID NO:37 in which X₄ is G or S, CDR2, comprising or consisting of SEQ ID NO:38 in which X₅ is A or T, and/or CDR3, comprising or consisting of SEQ ID NO:39 in which X₆ is Y or F, wherein the second single-domain antibody specifically binds CD123. In some aspects, the second single-domain antibody may comprise, or consist of, an amino acid sequence at least 80% identical, at least 85% identical, at least 90% identical, at least 95% identical, at least 97% identical, or at least 99% identical to SEQ ID NO:40, wherein the amino acid sequence comprises CDR1, comprising or consisting of SEQ ID NO:37 in which X₄ is G or S, CDR2, comprising or consisting of SEQ ID NO:38 in which X₅ is A or T, and/or CDR3, comprising or consisting of SEQ ID NO:39 in which X₆ is Y or F, and wherein the second single-domain antibody specifically binds CD123. In some aspects, the second single-domain antibody may comprise, or consist of, SEQ ID NO:40. In some aspects, the second single-domain antibody may comprise CDR1, comprising or consisting of any of SEQ ID NOs: 53, 57, 61, 65, 69, 73, and 77, CDR2, comprising or consisting of any of SEQ ID NOs: 54, 58, 62, 66, 70, 74, and 78, and/or CDR3, comprising or consisting of any of SEQ ID NOs: 55, 59, 63, 67, 71, 75, and 79, wherein the second single-domain antibody specifically binds Nectin-4. In some aspects, the second single-domain antibody may comprise, or consist of, an amino acid sequence at least 80% identical, at least 85% identical, at least 90% identical, at least 95% identical, at least 97% identical, or at least 99% identical to any of SEQ ID NOs: 56, 60, 64, 68, 72, 76, and 80, wherein the amino acid sequence comprises CDR1, comprising or consisting of any of SEQ ID NOs: 53, 57, 61, 65, 69, 73, and 77, CDR2, comprising or consisting of any of SEQ ID NOs: 54, 58, 62, 66, 70, 74, and 78, and/or CDR3, comprising or consisting of SEQ ID NOs: 55, 59, 63, 67, 71, 75, and 79, and wherein the second single-domain antibody specifically binds Nectin-4. In some aspects, the second single-domain antibody may comprise, or consist of, any of SEQ ID NOs: 56, 60, 64, 68, 72, 76, and 80.

One aspect of the disclosure is a reporter cell expressing a γδTCR and comprising a reporter gene responsive to activation of the reporter cell. In some aspects, the reporter cell is a reporter T cell. In some aspects, the reporter T cell is produced by modification of a Jurkat cell or a CTLL-2 T cell. In some aspects, the γδTCR may comprise a Vγ9 chain and/or a Vδ2 chain. In some aspects, the γδTCR may comprise a Vγ9Vδ2 TCR. In some aspects, the reporter gene may encode a reporter protein, which may be a fluorescent protein, a luminescent protein, a chemiluminescent protein, or an enzyme. In some aspects, the luminescent protein may be luciferase, which may be firefly luciferase, click beetle luciferase, marine copepod Gaussia luciferase, or deep-sea shrimp Nano luciferase. In some aspects, the reporter gene may comprise a nucleic acid molecule comprising a nucleotide sequence encoding the reporter protein operably linked to a promoter responsive to activation of the reporter cell, and, optionally, to one or more response elements. In some aspects, the promoter may be responsive to T cell activation. In some aspects, the promoter may be an NFAT promoter, an AP-1 promoter, an NFκB promoter, a FOXO promoter, a STAT3 promoter, a STATS promoter, or an IRF promoter. In one aspect, the reporter gene may comprise a nucleic acid molecule comprising a nucleotide sequence encoding the reporter protein under the control of a promoter and operably linked to one or more elements responsive to activation of the reporter cell. In some aspects, the promoter controlling the nucleotide sequence encoding the reporter protein may be a minimal promoter. In some aspects, the minimal promoter may be a TK minimal promoter, a CMV minimal promoter, an SV40 minimal promoter, or an 1EF1α minimal promoter. In some aspects, the response element may be selected from the group consisting of an NFAT gene response element, an AP-1 gene response element, an NFκB gene response element, a FOXO gene response element, a STAT3 gene response element, a STAT5 gene response element, or an IRF gene response element. In some aspects, the response elements may be arranged as tandem repeats (such as about any of 2, 3, 4, 5, 6, 7, 8, or more tandem repeats). In some aspects, the response element(s) may be positioned 5′ or 3′ to the reporter gene. In some aspects, the response element(s) may be located at a site 5′ from the promoter. In some aspects, the nucleic acid molecule may be stably integrated into the genome of the reporter cell. In one aspect, the reporter cell does not express the target antigen. In one aspect, the modified T cell does not express CD1d, CD40, CD123, PSMA, Nectin-4, or EGFR.

As noted herein, T cells may be used to produce reporter cells of the disclosure, thereby producing a reporter T cell. It will be apparent to one of skill in the art that because the cell used to produce the reporter T cell is a T cell, it may already express a T cell receptor, such as a T cell receptor having an alpha (α) chain and a (3) beta chain (i.e., an αβTCR). In some aspects, having a T-cell express both types of receptors (i.e., an αβTCR and a γδTCR) may not be desirable. Thus, in some aspects a reporter T cell, or a T cell used to produce the reporter T cell, may be engineered to eliminate expression of an αβTCR. Eliminating expression of αβTCR may be accomplished using any known method for silencing gene expression including “knocking-out” the genes encoding the TCR α chain protein and/or TCR β chain protein. Knocking out the α chain, or β chain gene may comprise making alterations, or deletion or insertion mutations at any location within the α chain loci, or β chain loci, or within genetic elements controlling expression of either or both loci. Methods of knocking-out a gene may comprise, for example, inserting a nucleic acid into a gene, deleting all or part of a gene, or interrupting, deleting, or editing a control element, such as a promoter. Methods of silencing genes within cells are known in the art. Thus, in one aspect, a reporter T cell of the disclosure may not express a functional TCR α chain or a functional TCR β chain. In one aspect, a reporter T cell of the disclosure may not express a functional TCR α chain and/or a functional TCR β chain. In one aspect, a reporter T cell of the disclosure may comprise a mutation within the endogenous α chain, or β chain locus or within genetic elements controlling expression of either or both loci, so that the reporter cell does not express a functional αβTCR.

One aspect of the disclosure is a reporter T cell expressing a γδTCR and comprising a T cell activation responsive reporter gene, wherein the reporter T cell does not express a functional αβTCR. In some aspects, the reporter T cell is a modified Jurkat cell or a modified CTLL-2 T cell. In some aspects, the γδTCR may comprise a Vγ9 chain and/or a Vδ2 chain. In some aspects, the γδTCR may comprise a Vγ9Vδ2 TCR. In some aspects, the reporter gene may be any gene that encodes a reporter protein, the presence or activity of which is detectable. In some aspects, the reporter gene may encode a reporter protein, which may be selected from the group consisting of a fluorescent protein, a luminescent protein, a chemiluminescent protein, and an enzyme. In some aspects, the luminescent protein may be luciferase, which may be firefly luciferase, click beetle luciferase, marine copepod Gaussia luciferase, or deep-sea shrimp Nano luciferase. In one aspect, the reporter gene may comprise a nucleic acid molecule comprising a nucleotide sequence encoding the reporter protein operably linked to a T cell activation responsive promoter, and, optionally, to one or more T cell activation response elements. In some aspects, the T cell activation responsive promoter may be selected from promoters known in the art to be responsive to T cell activation. In some aspects, the promoter may be an NFAT promoter, an AP-1 promoter, an NFκB promoter, a FOXO promoter, a STAT3 promoter, a STAT5 promoter, or an IRF promoter. In one aspect, the reporter gene may comprise a nucleic acid molecule comprising a nucleotide sequence encoding the reporter protein under the control of a promoter and operably linked to one or more T cell activation response elements. In some aspects, the promoter controlling the nucleotide sequence encoding the reporter protein may be a minimal promoter. In some aspects, the minimal promoter may be a TK minimal promoter, a CMV minimal promoter, an SV40 minimal promoter, or an 1EF1α minimal promoter. In some aspects, one or more T cell activation response elements may comprise a T cell activation response element selected from the group consisting of an NFAT gene response element, an AP-1 gene response element, an NFκB gene response element, a FOXO gene response element, a STAT3 gene response element, a STAT5 gene response element, or an IRF gene response element. In some aspects, the one or more T cell activation response elements may be arranged as tandem repeats (such as about any of 2, 3, 4, 5, 6, 7, 8, or more tandem repeats). In some aspects, one or more T cell activation response element(s) may be positioned 5′ or 3′ to the reporter gene. In some aspects, one or more T cell activation response element(s) may be located at a site 5′ from the promoter. In some aspects, the nucleic acid molecule may be stably integrated into the T cell genome. In one aspect, the modified T cell may comprise one or more knock out mutations within its α chain loci, or β chain loci, or within genetic elements controlling expression of either or both loci. In one aspect, the modified T cell lacks a functional α chain and/or β chain gene. In one aspect, the modified T cell does not express a target antigen expressed by a target cell in an assay in which the T cell is intended for use. In one aspect, the modified T cell does not express CD1d, CD40, CD123, PSMA, Nectin-4, or EGFR.

In some aspects, a cell used to produce a reporter cell of the disclosure may be engineered to express a γδTCR. In some aspects, a cell may be engineered to express a γδTCR by introducing into the cell one or more exogenous nucleic acid molecules that encode the γ chain protein and/or the δ chain protein of a TCR. The one or more exogenous nucleic acids may, but need not, be stably inserted into the genome of the cell. Expression of the γ chain and/or δ chain proteins of a TCR may be placed under the control of any endogenous, exogenous, or heterologous promoter capable of driving expression of a gene in the cell. Examples of suitable promoters for driving expression of the encoded γ chain gene and/or δ chain gene included, but are not limited to, a thymidine kinase (TK) promoter, a cytomegalovirus (CMV) promoter, a simian virus (SV) 40 promoter.

One aspect of the disclosure is a reporter cell expressing a γδTCR and comprising a reporter gene responsive to activation of the reporter cell, wherein the reporter cell comprises one or more exogenous nucleic acid molecules that encode the TCR γ and δ chain proteins. One aspect of the disclosure is a reporter T cell expressing a γδTCR and comprising a T cell activation responsive reporter gene, wherein the reporter T cell comprises one or more exogenous nucleic acid molecules that encode the TCR γ and δ chain proteins, and wherein the reporter T cell does not express a functional αβTCR. In some aspects, the reporter T cell is a modified Jurkat cell or a modified CTLL-2 T cell. In some aspects, the γδTCR may comprise a Vγ9 chain and/or a Vδ2 chain. In some aspects, the γδTCR may comprise a Vγ9Vδ2 TCR. In some aspects, the reporter gene may be any gene that encodes a reporter protein, the presence or activity of which is detectable. In some aspects, the reporter protein may be a fluorescent protein, a luminescent protein, a chemiluminescent protein, or an enzyme, such as alkaline phosphatase or beta-galactosidase. In some aspects, the luminescent protein may be luciferase, which may be firefly luciferase, click beetle luciferase, marine copepod Gaussia luciferase, or deep-sea shrimp Nano luciferase. In some aspects, the reporter gene comprises a nucleic acid molecule comprising a nucleotide sequence encoding the reporter protein operably linked to a promoter responsive to activation of the reporter cell, and, optionally, to one or more response elements. In some aspects, the promoter may be selected from promoters known in the art to be responsive to activation of the reporter cell. In some aspects, the promoter may be an NFAT promoter, an AP-1 promoter, an NFκ3 promoter, a FOXO promoter, a STAT3 promoter, a STAT5 promoter, or an IRF promoter. In one aspect, the reporter gene comprises a nucleic acid molecule comprising a nucleotide sequence encoding the reporter protein under the control of a promoter and operably linked to one or more response element. In some aspects, the promoter controlling the nucleotide sequence encoding the reporter protein may be a minimal promoter. In some aspects, the minimal promoter may be a TK minimal promoter, a CMV minimal promoter, an SV40 minimal promoter, or an 1EF1α minimal promoter. In some aspects, the one or more T cell activation response elements may comprise a T cell activation response element selected from the group consisting of an NFAT gene response element, an AP-1 gene response element, an NFκB gene response element, a FOXO gene response element, a STAT3 gene response element, a STAT5 gene response element, or an IRF gene response element. In some aspects, the one or more response elements may be arranged as tandem repeats (such as about any of 2, 3, 4, 5, 6, 7, 8, or more tandem repeats). In some aspects, the one or more response element(s) may be positioned 5′ or 3′ to the reporter gene. In some aspects, the one or more response element(s) may be located at a site 5′ from the promoter. In some aspects, the nucleic acid molecule may be stably integrated into the genome of the reporter cell. In some aspects, the one or more exogenous nucleic acid molecule(s) may be stably integrated into the T cell genome. In one aspect, the reporter cell may comprise one or more knock out mutations within its α chain loci, or β chain loci, or within genetic elements controlling expression of either or both loci.

In one aspect, the reporter cell lacks a functional α chain and/or β chain gene. In one aspect, the reporter cell does not express a target antigen expressed by a target cell in an assay in which the reporter is intended for use. In one aspect, the modified T cell does not express CD1d, CD40, CD123, PSMA, Nectin-4, or EGFR. In some embodiments, the reporter cells of the present disclosure may comprise one or more modifications that reduces the expression the target antigen (or a protein that immunologically cross-reacts with the target protein). Thus, in these methods, a reporter cell of the disclosure may not express the target antigen or a molecule that immunologically cross-reacts with the target antigen. For example, a reporter cell of the disclosure may not express an antigen selected from the group consisting of CD1d, CD40, CD123, PSMA, Nectin-4, and EGFR. Such embodiments are useful for reducing a background signal resulting from engagement of the target-antigen binding site of the γδ-TDbAb by the target antigen expressed on the reporter cell.

In some embodiments, the reporter cell of the present disclosure comprises one or more genomic DNA modifications that eliminates, or substantially reduces, expression of the target antigen (e.g., a genetic knock-out). Systems for generating genetic knock-outs are known in the art, for example, the Cre-Lox or FLP-FRT recombination systems, TALENs, zinc finger nucleases, and endonucleases. In some embodiments, an endonuclease is used to eliminate expression of the target antigen. Exemplary endonucleases are known in the art, for example Cas endonucleases for use with a guide RNA in the CRISPR-Cas system. In some embodiments, the reporter cell comprises a Cas endonuclease and guide RNA that binds to a target sequence in the target antigen gene (e.g., a target sequence in the CD1d gene, the PSMA gene, the CD40 gene, the EGFR gene, the CD123 gene, and/or the Nectin 4 gene). In some embodiments, the reporter cell of the present disclosure may comprise an oligonucleotide, such as an siRNA or shRNA, that inhibits translation of the mRNA encoding the target antigen.

In some embodiments, the reporter cell of the present disclosure stably expresses a Cas endonuclease (e.g., a Cas9 endonuclease). In such embodiments, expression of a target antigen can be eliminated by selection of the appropriate guide RNA (gRNA) and introduction of the gRNA into the reporter cell. Reporter cells can then be assayed by means known in the art (e.g., flow cytometry, Western Blot, etc.) and selected for successful knock-out of the target antigen. Selected knock-out reporter cells can be expanded for use according to the methods described herein.

One aspect of the disclosure is a reporter T cell expressing a γδTCR and comprising a reporter gene responsive to T cell activation, wherein the reporter T cell comprises one or more exogenous nucleic acid molecules that encode the TCR γ and δ chain proteins, wherein the reporter T cell does not express a functional αβTCR, and wherein the reporter T-cell does not express a target antigen. In some aspects, the reporter T cell is a modified Jurkat cell or a modified CTLL-2 T cell. In some aspects, the γδTCR may comprise a Vγ9 chain and/or a Vδ2 chain. In some aspects, the γδTCR may comprise a Vγ9Vδ2 TCR. In some aspects, the reporter gene may be any gene that encodes a reporter protein, the presence or activity of which is detectable. In some aspects, the reporter protein may be a fluorescent protein, a luminescent protein, a chemiluminescent protein, or an enzyme, such as alkaline phosphatase or beta-galactosidase. In some aspects, the luminescent protein may be luciferase, which may be firefly luciferase, click beetle luciferase, marine copepod Gaussia luciferase, or deep-sea shrimp Nano luciferase. In one aspect, the reporter gene comprises a nucleic acid molecule comprising a nucleotide sequence encoding the reporter protein operably linked to a T cell activation responsive promoter, and, optionally, to one or more T cell activation response elements. In some aspects, the T cell activation responsive promoter may be selected from promoters known in the art to be responsive to T cell activation. In some aspects, the promoter may be an NFAT promoter, an AP-1 promoter, an NFκB promoter, a FOXO promoter, a STAT3 promoter, a STAT5 promoter, or an IRF promoter. In one aspect, the reporter gene comprises a nucleic acid molecule comprising a nucleotide sequence encoding the reporter protein under the control of a promoter and operably linked to one or more T cell activation response elements. In some aspects, the promoter may be a minimal promoter. In some aspects, the minimal promoter may be a TK minimal promoter, a CMV minimal promoter, an SV40 minimal promoter, or an 1EF1α minimal promoter. In some aspects, the T cell activation response element may comprise a T cell activation response element selected from the group consisting of an NFAT gene response element, an AP-1 gene response element, an NFκB gene response element, a FOXO gene response element, a STAT3 gene response element, a STAT5 gene response element, or an IRF gene response element. In some aspects, the one or more T cell activation response elements may be arranged as tandem repeats (such as about any of 2, 3, 4, 5, 6, 7, 8, or more tandem repeats). In some aspect, the one or more T cell activation response element(s) may be positioned 5′ or 3′ to the reporter gene. In some aspects, the one or more T cell activation response element(s) may be located at a site 5′ from the promoter. In some aspects, the nucleic acid molecule may be stably integrated into the T cell genome. In some aspects, the one or more exogenous nucleic acid molecule(s) may be stably integrated into the T cell genome. In one aspect, the modified T cell may comprise one or more knock out mutations within its α chain loci, or β chain loci, or within genetic elements controlling expression of either or both loci. In one aspect, the modified T cell lacks a functional α chain gene and/or β chain gene. In one aspect, the modified T cell does not express a target antigen, or a molecule that immunologically cross-reacts with a target antigen, expressed by a target cell in an assay in which the T cell is intended for use. In one aspect, the modified T cell does not express CD1d, CD40, CD123, PSMA, Nectin-4, or EGFR.

In some aspects, the knockout or knockdown of the target gene removes the background signal from the signal. “Background signal,” or “background noise,” is the signal detected when the receptor cells are incubated with the γδ-TDMBC not in the presence of a target cell.

TABLE 1
Sequence listing

| SEQ | | | |
|---|---|---|---|
| ID | code | Description | Sequence |
| 1 | 5C8 | CDR1 | NYAMG |
| 2 | 5C8 | CDR2 | AISWSGGSTSYADSVKG |
| 3 | 5C8 | CDR3 | QFSGADYGFGRLGIRGYEYDY |
| 4 | 5C8 | VHH | EVQLVESGGGLVQAGGSLRLSCAASGRPFS |
| | | | NYAMGWFRQAPGKEREFVAAISWSGGSTS |
| | | | YADSVKGRFTISRDNAKNTVYLQMNSPKP |
| | | | EDTAIYYCAAQFSGADYGFGRLGIRGYEYD |
| | | | YWGQGTQVTVSS |
| 5 | 5C8 var | CDR1 | NYAMX₁, wherein X₁ is S or G |
| 6 | 5C8 var | CDR2 | AISWSGGSTSY ADSVKG |
| 7 | 5C8 var | CDR3 | QFSGADYGFGRLGIRGYEYDY |
| 8 | 5C8 var1 | CDR1 | NYAMS |
| 9 | 5C8 var1 | CDR2 | AISWSGGSTSYADSVKG |
| 10 | 5C8 var1 | CDR3 | QFSGADYGFGRLGIRGYEYDY |
| 11 | 5C8var1 | VHH | EVQLLESGGGSVQPGGSLRLSCAASGRPFS |
| | | | NYAMSWFRQAPGKEREFVSAISWSGGSTS |
| | | | YADSVKGRFTISRDNSKNTLYLQMNSLRAE |
| | | | DTAVYYCAAQFSGADYGFGRLGIRGYEYD |
| | | | YWGQGTQVTVSS |
| 12 | 5C8 var | CDR1 | NYAMX₁, wherein X₁ is S or G |
| | (Y105 + R109) | | |
| 13 | 5C8 var | CDR2 | AISWSGGSTSYADSVKG |
| | (Y105 + R109) | | |
| 14 | 5C8 var | CDR3 | QFSGADX₂GFGX₃LGIRGYEYDY, wherein X₂ |
| | (Y105 + R109) | | can be any amino acid and wherein X₃ is not R |
| 15 | 5C8 var | CDR3 | QFSGADX₂GFGX₃GYEYDY, wherein X₂ can |
| | (Y105 + R109) | | be any amino acid and wherein X₃ is not R |
| | ΔLGIR | | |
| 16 | 5C8 var | VHH | EVQLLESGGGSVQPGGSLRLSCAASGRPFS |
| | (Y105 + R109) | | NYAMX₁WFRQAPGKEREFVSAISWSGGSTS |
| | | | YADSVKGRFTISRDNSKNTLYLQMNSLRAE |
| | | | DTAVYYCAAQFSGADX₂GFGX₃LGIRGYEY |
| | | | DYWGQGTQVTVSS, wherein X₁ is S or G, |
| | | | wherein X₂ can be any amino acid and wherein |
| | | | X₃ is not R |
| 17 | 5C8 var1 | CDR3 | QFSGADFGFGALGIRGYEYDY |
| | (Y105F + R109A) | | |
| 18 | 5C8 var1 | CDR3 | QFSGADSGFGALGIRGYEYDY |
| | (Y105S + R109A) | | |
| 19 | 5C8 var1 | CDR3 | QFSGADFGFGKLGIRGYEYDY |
| | (Y105F + R109K) | | |
| 20 | 5C8 var1 | CDR3 | QFSGADSGFGKLGIRGYEYDY |
| | (Y105S + R109K) | | |
| 21 | 5C8 var1 | CDR3 | QFSGADFGFGAGYEYDY |
| | (Y105F + R109A) | | |
| | ΔLGIR | | |
| 22 | 5C8 var1 | CDR3 | QFSGADSGFGAGYEYDY |
| | (Y105S + R109A) | | |
| | ΔLGIR | | |
| 23 | 5C8 var1 | CDR3 | QFSGADFGFGKGYEYDY |
| | (Y105F + R109K) | | |
| | ΔLGIR | | |
| 24 | 5C8 var1 | CDR3 | QFSGADSGFGKGYEYDY |
| | (Y105S + R109K) | | |
| | ΔLGIR | | |
| 25 | 5C8 var1 | VHH | EVQLLESGGGSVQPGGSLRLSCAASGRPFS |
| | (Y105F + R109A) | | NYAMSWFRQAPGKEREFVSAISWSGGSTS |
| | | | YADSVKGRFTISRDNSKNTLYLQMNSLRAE |
| | | | DTAVYYCAAQFSGADFGFGALGIRGYEYD |
| | | | YWGQGTQVTVSS |
| 26 | 5C8 var1 | VHH | EVQLLESGGGSVQPGGSLRLSCAASGRPFS |
| | (Y105S + R109A) | | NYAMSWFRQAPGKEREFVSAISWSGGSTS |
| | | | YADSVKGRFTISRDNSKNTLYLQMNSLRAE |
| | | | DTAVYYCAAQFSGADSGFGALGIRGYEYD |
| | | | YWGQGTQVTVSS |
| 27 | 5C8 var1 | VHH | EVQLLESGGGSVQPGGSLRLSCAASGRPFS |
| | (Y105F + R109K) | | NYAMSWFRQAPGKEREFVSAISWSGGSTS |
| | | | YADSVKGRFTISRDNSKNTLYLQMNSLRAE |
| | | | DTAVYYCAAQFSGADFGFGKLGIRGYEYD |
| | | | YWGQGTQVTVSS |
| 28 | 5C8 var1 | VHH | EVQLLESGGGSVQPGGSLRLSCAASGRPFS |
| | (Y105S + R109K) | | NYAMSWFRQAPGKEREFVSAISWSGGSTS |
| | | | YADSVKGRFTISRDNSKNTLYLQMNSLRAE |
| | | | DTAVYYCAAQFSGADSGFGKLGIRGYEYD |
| | | | YWGQGTQVTVSS |
| 29 | 5C8 var1 | VHH | EVQLLESGGGSVQPGGSLRLSCAASGRPFS |
| | (Y105F + R109A) | | NYAMSWFRQAPGKEREFVSAISWSGGSTS |
| | ΔLGIR | | YADSVKGRFTISRDNSKNTLYLQMNSLRAE |
| | | | DTAVYYCAAQFSGADFGFGAGYEYDYWG |
| | | | QGTQVTVSS |
| 30 | 5C8 var1 | VHH | EVQLLESGGGSVQPGGSLRLSCAASGRPFS |
| | (Y105S + R109A) | | NYAMSWFRQAPGKEREFVSAISWSGGSTS |
| | ΔLGIR | | YADSVKGRFTISRDNSKNTLYLQMNSLRAE |
| | | | DTAVYYCAAQFSGADSGFGAGYEYDYWG |
| | | | QGTQVTVSS |
| 31 | 5C8 var1 | VHH | EVQLLESGGGSVQPGGSLRLSCAASGRPFS |
| | (Y105F + R109K) | | NYAMSWFRQAPGKEREFVSAISWSGGSTS |
| | ΔLGIR | | YADSVKGRFTISRDNSKNTLYLQMNSLRAE |
| | | | DTAVYYCAAQFSGADFGFGKGYEYDYWG |
| | | | QGTQVTVSS |
| 32 | 5C8 var1 | VHH | EVQLLESGGGSVQPGGSLRLSCAASGRPFS |
| | (Y105S + R109K) | | NYAMSWFRQAPGKEREFVSAISWSGGSTS |
| | ΔLGIR | | YADSVKGRFTISRDNSKNTLYLQMNSLRAE |
| | | | DTAVYYCAAQFSGADSGFGKGYEYDYWG |
| | | | QGTQVTVSS |
| 33 | 7D12 (EGFR) | CDR1 | SYGMG |
| 34 | 7D12 (EGFR) | CDR2 | GISWRGDSTGYADSVKG |
| 35 | 7D12 (EGFR) | CDR3 | AAGSAWYGTLYEYDY |
| 36 | 7D12var8 (EGFR) | VHH | EVQLVESGGGSVQPGGSLRLSCAASGRTSR |
| | | | SYGMGWFRQAPGKEREFVSGISWRGDSTG |
| | | | YADSVKGRFTISRDNAKNTVDLQMNSLRA |
| | | | EDTAVYYCAAAAGSAWYGTLYEYDYWG |
| | | | QGTLVTVSS |
| 37 | 1D2 (CD123) | CDR1 | GRTASSYVMX₄, wherein X₄ is G or S |
| 38 | 1D2 (CD123) | CDR2 | VINWNGDSTYYX₅DSVKG, wherein X₅ is A or |
| | | | T |
| 39 | 1D2 (CD123) | CDR3 | DTRREWYRDGX₆WGPPARYEYDY, wherein |
| | | | X₆ is Y or F |
| 40 | 1D2 (CD123) | VHH | EVQLVESGGGLVQAGGSLRLSCAASGRTA |
| | | | SSYVMGWFRQAPGKEREFVAVINWNGDST |
| | | | YYTDSVKGRFAISRDNAKNTVYLQMNSLK |
| | | | PEDTAVYYCAADTRREWYRDGYWGPPAR |
| | | | YEYDYRGQGTQVTVSS |
| 41 | LV1050 (PSMA) | CDR1 | RFMISEYSMH |
| 42 | LV1050 (PSMA) | CDR2 | TINPAGTTDYADSVKG |
| 43 | LV1050 (PSMA) | CDR3 | DGYGY |
| 44 | LV1050 (PSMA) | VHH | EVQLVESGGGLVQPGGSLRLSCAASRFMIS |
| | | | EYSMHWVRQAPGKGLEWVSTINPAGTTDY |
| | | | ADSVKGRFTISRDNAKNTLYLQMNSLRAE |
| | | | DTAVYYCDGYGYRGQGTQVTVSS |
| 45 | 1D12 (CD1d) | CDR1 | DNVMG |
| 46 | 1D12 (CD1d) | CDR2 | TIRTGGSTNYADSVKG |
| 47 | 1D12 (CD1d) | CDR3 | TIPVPSTPYDY |
| 48 | 1D12 (CD1d) | VHH | QVQLVESGGGLVQAGGSLRLSCAASGSMF |
| | | | SDNVMGWYRQAPGKQRELVATIRTGGSTN |
| | | | YADSVKGRFTISRDNAKNTVYLQMNSLKP |
| | | | EDTAVYYCRHTIPVPSTPYDYWGQGTQVT |
| | | | VSS |
| 49 | v19 (CD40) | CDR1 | RSAMG |
| 50 | v19 (CD40) | CDR2 | AIGTRGGSTKYADSVKG |
| 51 | v19 (CD40) | CDR3 | RGPGYPSAAIFQDEYHY |
| 52 | v19 (CD40) | VHH | QVQLQESGGGLVQAGGSLRLSCAASGRTF |
| | | | GRSAMGWFRQAPGKEREFVAAIGTRGGST |
| | | | KYADSVKGRFTISTDNASNTVYLQMDSLKP |
| | | | EDTAVYRCAVRGPGYPSAAIFQDEYHYWG |
| | | | QGTQVTVSS |
| 53 | LV1184 (Nectin 4) | CDR1 | INLMG |
| 54 | LV1184 (Nectin 4) | CDR2 | SISPGGSVRYADSVKG |
| 55 | LV1184 (Nectin 4) | CDR3 | ESERTYYFDS |
| 56 | LV1184 (Nectin 4) | VHH | EVQLVESGGGLVQAGGSLRLSCAASGSISSI |
| | | | NLMGWFRQAPAKQRELVTSISPGGSVRYA |
| | | | DSVKGRFTISRDVAKNTVDLQMNSLKPEDT |
| | | | AVYYCAAESERTYYFDSWGQGTQVTVSS |
| 57 | LV1181 (Nectin 4) | CDR1 | TITRGGSTNYADSVKG |
| 58 | LV1181 (Nectin 4) | CDR2 | VEPSGMGWRDY |
| 59 | LV1181 (Nectin 4) | CDR3 | EVQLVESGGGLVQAGGSLRLSCAASGSISS |
| 60 | LV1181 (Nectin 4) | VHH | EVQLVESGGGLVQAGGSLRLSCAASGSISSI |
| | | | NLMGWHRQAPGKQRELVATITRGGSTNYA |
| | | | DSVKGRFTISRDNGKNTVYLQMNSLKPEDT |
| | | | AAYYCVDVEPSGMGWRDYWGQGTQVTV |
| | | | SS |
| 61 | LV1185 (Nectin 4) | CDR1 | LNIMG |
| 62 | LV1185 (Nectin 4) | CDR2 | TITTGGSTNYADSVRG |
| 63 | LV1185 (Nectin 4) | CDR3 | ELVRRGPTTY |
| 64 | LV1185 (Nectin 4) | VHH | EVQLVESGGGLVQAGGSLRLSCAASRSTFS |
| | | | LNIMGWYRQAPGKQREYVATITTGGSTNY |
| | | | ADSVRGRFTISRDNAEDTVYLQMNSLKPED |
| | | | TAVYYCTAELVRRGPTTYWGRGTQVTVSS |
| 65 | LV1186 (Nectin 4) | CDR1 | LNIMG |
| 66 | LV1186 (Nectin 4) | CDR2 | TITSGGSTNYADSVRG |
| 67 | LV1186 (Nectin 4) | CDR3 | ELVRRGPTTY |
| 68 | LV1186 (Nectin 4) | VHH | EVQLVESGGGLVQAGGSLRLSCAASRSTFS |
| | | | LNIMGWYRQAPGKQREYVATITSGGSTNY |
| | | | ADSVRGRFTISRDNAENTVYLQMDSLKPED |
| | | | TAVYYCTAELVRRGPTTYWGRGTQVTVSS |
| 69 | LV1178 (Nectin 4) | CDR1 | RLTMG |
| 70 | LV1178 (Nectin 4) | CDR2 | RVYASGGLTDYADSVKG |
| 71 | LV1178 (Nectin 4) | CDR3 | GLWADMRTMTSTRGY |
| 72 | LV1178 (Nectin 4) | VHH | EVQLVESGGALVQAGGSLRLSCSVSGLTASRLT |
| | | | MGWFRQAPGKEREFVARVYASGGLTDYADSVKG |
| | | | RFTISRDNAKNTVYLQMNSLEPEDTAVYYCVAG |
| | | | LWADMRTMTSTRGYWGQGTQVTVSS |
| 73 | LV1183 (Nectin 4) | CDR1 | INLMG |
| 74 | LV1183 (Nectin 4) | CDR2 | SITRGGSTWYVDSVKG |
| 75 | LV1183 (Nectin 4) | CDR3 | ESIGSYTFDR |
| 76 | LV1183 (Nectin 4) | VHH | EVQLVESGGGLVQAGGSLRLSCAASGSIFDINL |
| | | | MGWYRQAPGKLRELVASITRGGSTWYVDSVKGR |
| | | | FIISRDSAKNTVYLQMNSLKPGDTAVYYCAAES |
| | | | IGSYTFDRWGQGTQVTVSS |
| 77 | LV1187 (Nectin 4) | CDR1 | INVMG |
| 78 | LV1187 (Nectin 4) | CDR2 | GITSGGSRRLADSVKG |
| 79 | LV1187 (Nectin 4) | CDR3 | EAVVGDEPY |
| 80 | LV1187 (Nectin 4) | VHH | EVQLVESGGGLVQAGGFLRLSCAASRSVFRINV |
| | | | MGWYRQAPGKQRELVAGITSGGSRRLADSVKGR |
| | | | FTISRDNAKNTVYLQMNSLKPEDTAVYYCFAEA |
| | | | VVGDEPYWGQGTQVTVSS |
| 81 | linker | | GGGGS |
| 82 | | Modified | AAASDKTHTCPPCP |
| | | hinge | |
| 83 | IgG1 | Heavy chain | AAASDKTHTCPPCPAPEFEGGPSVFLFPPKP |
| | L234F | constant | KDTLMISRTPEVTCVVVDVSHEDPEVKFN |
| | L235E | region variant | WYVDGVEVHNAKTKPREEQYNSTYRVVS |
| | T366W | | VLTVLHQDWLNGKEYKCKVSNKALPAPIE |
| | | | KTISKAKGQPREPQVYTLPPSRDELTKNQV |
| | | | SLWCLVKGFYPSDIAVEWESNGQPENNYK |
| | | | TTPPVLDSDGSFFLYSKLTVDKSRWQQGNV |
| | | | FSCSVMHEALHNHYTQKSLSLSPGX, |
| | | | wherein X is a K that may or may not be present |
| 84 | IgG1 | Heavy chain | AAASDKTHTCPPCPAPEFEGGPSVFLFPPKP |
| | L234F | constant | KDTLMISRTPEVTCVVVDVSHEDPEVKFN |
| | L235E | region variant | WYVDGVEVHNAKTKPREEQYNSTYRVVS |
| | T366S | | VLTVLHQDWLNGKEYKCKVSNKALPAPIE |
| | L368A | | KTISKAKGQPREPQVYTLPPSRDELTKNQV |
| | Y407V | | SLSCAVKGFYPSDIAVEWESNGQPENNYKT |
| | | | TPPVLDSDGSFFLVSKLTVDKSRWQQGNVF |
| | | | SCSVMHEALHNHYTQKSLSLSPGX, wherein |
| | | | X is a K that may or may not be present |
| 85 | 7D12var8-Fc | VHH-Fc | EVQLVESGGGSVQPGGSLRLSCAASGRTSR |
| | | | SYGMGWFRQAPGKEREFVSGISWRGDSTG |
| | | | YADSVKGRFTISRDNAKNTVDLQMNSLRA |
| | | | EDTAVYYCAAAAGSAWYGTLYEYDYWG |
| | | | QGTLVTVSSAAASDKTHTCPPCPAPEFEGG |
| | | | PSVFLFPPKPKDTLMISRTPEVTCVVVDVSH |
| | | | EDPEVKFNWYVDGVEVHNAKTKPREEQY |
| | | | NSTYRVVSVLTVLHQDWLNGKEYKCKVS |
| | | | NKALPAPIEKTISKAKGQPREPQVYTLPPSR |
| | | | DELTKNQVSLSCAVKGFYPSDIAVEWESNG |
| | | | QPENNYKTTPPVLDSDGSFFLVSKLTVDKS |
| | | | RWQQGNVFSCSVMHEALHNHYTQKSLSLS |
| | | | PGX, wherein X is a K that may or may not be |
| | | | present |
| 86 | 1D2-Fc | VHH-Fc | EVQLVESGGGLVQAGGSLRLSCAASGRTA |
| | | | SSYVMGWFRQAPGKEREFVAVINWNGDST |
| | | | YYTDSVKGRFAISRDNAKNTVYLQMNSLK |
| | | | PEDTAVYYCAADTRREWYRDGYWGPPAR |
| | | | YEYDYRGQGTQVTVSSAAASDKTHTCPPC |
| | | | PAPEFEGGPSVFLFPPKPKDTLMISRTPEVT |
| | | | CVVVDVSHEDPEVKFNWYVDGVEVHNAK |
| | | | TKPREEQYNSTYRVVSVLTVLHQDWLNGK |
| | | | EYKCKVSNKALPAPIEKTISKAKGQPREPQ |
| | | | VYTLPPSRDELTKNQVSLSCAVKGFYPSDI |
| | | | AVEWESNGQPENNYKTTPPVLDSDGSFFLV |
| | | | SKLTVDKSRWQQGNVFSCSVMHEALHNH |
| | | | YTQKSLSLSPGX, wherein X is a K that may or |
| | | | may not be present |
| 87 | 5C8var1 | VHH-Fc | EVQLLESGGGSVQPGGSLRLSCAASGRPFS |
| | (Y105F R109A)-Fc | | NYAMSWFRQAPGKEREFVSAISWSGGSTS |
| | | | YADSVKGRFTISRDNSKNTLYLQMNSLRAE |
| | | | DTAVYYCAAQFSGADFGFGALGIRGYEYD |
| | | | YWGQGTQVTVSSAAASDKTHTCPPCPAPE |
| | | | FEGGPSVFLFPPKPKDTLMISRTPEVTCVVV |
| | | | DVSHEDPEVKFNWYVDGVEVHNAKTKPR |
| | | | EEQYNSTYRVVSVLTVLHQDWLNGKEYKC |
| | | | KVSNKALPAPIEKTISKAKGQPREPQVYTLP |
| | | | PSRDELTKNQVSLWCLVKGFYPSDIAVEWE |
| | | | SNGQPENNYKTTPPVLDSDGSFFLYSKLTV |
| | | | DKSRWQQGNVFSCSVMHEALHNHYTQKS |
| | | | LSLSPGX, wherein X is a K that may or may not |
| | | | be present |
| 88 | 5C8var1 | VHH-Fc | EVQLLESGGGSVQPGGSLRLSCAASGRPFS |
| | (Y105S R109A)-Fc | | NYAMSWFRQAPGKEREFVSAISWSGGSTS |
| | | | YADSVKGRFTISRDNSKNTLYLQMNSLRAE |
| | | | DTAVYYCAAQFSGADSGFGALGIRGYEYD |
| | | | YWGQGTQVTVSSAAASDKTHTCPPCPAPE |
| | | | FEGGPSVFLFPPKPKDTLMISRTPEVTCVVV |
| | | | DVSHEDPEVKFNWYVDGVEVHNAKTKPR |
| | | | EEQYNSTYRVVSVLTVLHQDWLNGKEYKC |
| | | | KVSNKALPAPIEKTISKAKGQPREPQVYTLP |
| | | | PSRDELTKNQVSLWCLVKGFYPSDIAVEWE |
| | | | SNGQPENNYKTTPPVLDSDGSFFLYSKLTV |
| | | | DKSRWQQGNVFSCSVMHEALHNHYTQKS |
| | | | LSLSPGX, wherein X is a K that may or may not |
| | | | be present |
| 89 | 5C8var1 | VHH-Fc | EVQLLESGGGSVQPGGSLRLSCAASGRPFS |
| | (Y105F R109K)-Fc | | NYAMSWFRQAPGKEREFVSAISWSGGSTS |
| | | | YADSVKGRFTISRDNSKNTLYLQMNSLRAE |
| | | | DTAVYYCAAQFSGADFGFGKLGIRGYEYD |
| | | | YWGQGTQVTVSSAAASDKTHTCPPCPAPE |
| | | | FEGGPSVFLFPPKPKDTLMISRTPEVTCVVV |
| | | | DVSHEDPEVKFNWYVDGVEVHNAKTKPR |
| | | | EEQYNSTYRVVSVLTVLHQDWLNGKEYKC |
| | | | KVSNKALPAPIEKTISKAKGQPREPQVYTLP |
| | | | PSRDELTKNQVSLWCLVKGFYPSDIAVEWE |
| | | | SNGQPENNYKTTPPVLDSDGSFFLYSKLTV |
| | | | DKSRWQQGNVFSCSVMHEALHNHYTQKS |
| | | | LSLSPGX, wherein X is a K that may or may not |
| | | | be present |
| 90 | 5C8var1 | VHH-Fc | EVQLLESGGGSVQPGGSLRLSCAASGRPFS |
| | (Y105S R091K)-Fc | | NYAMSWFRQAPGKEREFVSAISWSGGSTS |
| | | | YADSVKGRFTISRDNSKNTLYLQMNSLRAE |
| | | | DTAVYYCAAQFSGADSGFGKLGIRGYEYD |
| | | | YWGQGTQVTVSSAAASDKTHTCPPCPAPE |
| | | | FEGGPSVFLFPPKPKDTLMISRTPEVTCVVV |
| | | | DVSHEDPEVKFNWYVDGVEVHNAKTKPR |
| | | | EEQYNSTYRVVSVLTVLHQDWLNGKEYKC |
| | | | KVSNKALPAPIEKTISKAKGQPREPQVYTLP |
| | | | PSRDELTKNQVSLWCLVKGFYPSDIAVEWE |
| | | | SNGQPENNYKTTPPVLDSDGSFFLYSKLTV |
| | | | DKSRWQQGNVFSCSVMHEALHNHYTQKS |
| | | | LSLSPGX, wherein X is a K that may or may not |
| | | | be present |
| 91 | 1D2 var1 | VHH | EVQLVESGGGLVQPGGSLRLSCAASGRTAS |
| | | | SYVMGWFRQAPGKEREFVSVINWNGDSTY |
| | | | YADSVKGRFTISRDNSKNTLYLQMNSLRAE |
| | | | DTAVYYCAADTRREWYRDGYWGPPARYE |
| | | | YDYRGQGTQVTVSS |
| 92 | 1D2 var2 | VHH | EVQLVESGGGLVQPGGSLRLSCAASGRTAS |
| | | | SYVMGWVRQAPGKEREWVSVINWNGDST |
| | | | YYADSVKGRFTISRDNSKNTLYLQMNSLR |
| | | | AEDTAVYYCAADTRREWYRDGYWGPPAR |
| | | | YEYDYRGQGTLVTVSS |
| 93 | 1D2 var3 | VHH | EVQLVESGGGLVQPGGSLRLSCAASGRTAS |
| | | | SYVMSWFRQAPGKEREWVAVINWNGDST |
| | | | YYADSVKGRFTISRDNSKNTVYLQMNSLR |
| | | | AEDTAVYYCAADTRREWYRDGYWGPPAR |
| | | | YEYDYRGQGTLVTVSS |
| 94 | 1D2 var4 | VHH | EVQLVESGGGVVQPGGSLRLSCAASGRTAS |
| | | | SYVMSWFRQAPGKEREWVAVINWNGDST |
| | | | YYADSVKGRFTISRDNSKNTLYLQMNSLR |
| | | | AEDTAVYYCAADTRREWYRDGYWGPPAR |
| | | | YEYDYRGQGTTVTVSS |
| 95 | 1D2 var5 | VHH | EVQLVESGGGLVQPGGSLRLSCAASGRTAS |
| | | | SYVMSWFRQAPGKEREFVAVINWNGDSTY |
| | | | YADSVKGRFTISRDNSKNTVYLQMNSLRA |
| | | | EDTAVYYCAADTRREWYRDGYWGPPARY |
| | | | EYDYRGQGTLVTVSS |
| 96 | 1D2 var6 | VHH | EVQLVESGGGLVQPGGSLRLSCAASGRTAS |
| | | | SYVMGWVRQAPGKGLEWVSVINWNGDST |
| | | | YYADSVKGRFTISRDNSKNTLYLQMNSLR |
| | | | AEDTAVYYCAADTRREWYRDGYWGPPAR |
| | | | YEYDYRGQGTLVTVSS |
| 97 | 1D2 var7 | VHH | EVQLVESGGGLVQPGGSLRLSCAASGRTAS |
| | | | SYVMSWVRQAPGKGLEWVSVINWNGDST |
| | | | YYADSVKGRFTISRDNSKNTLYLQMNSLR |
| | | | AEDTAVYYCAADTRREWYRDGYWGPPAR |
| | | | YEYDYRGQGTLVTVSS |
| 98 | 1D2 var8 | VHH | EVQLVESGGGLVQPGGSLRLSCAASGRTAS |
| | | | SYVMSWFRQAPGKGLEWVAVINWNGDST |
| | | | YYADSVKGRFTISRDNSKNTVYLQMNSLR |
| | | | AEDTAVYYCAADTRREWYRDGYWGPPAR |
| | | | YEYDYRGQGTLVTVSS |
| 99 | 1D2 var9 | VHH | EVQLVESGGGLVQAGGSLRLSCAASGRTA |
| | | | SSYVMGWFRQAPGKEREWVAVINWNGDS |
| | | | TYYTDSVKGRFAISRDNAKNTVYLQMNSL |
| | | | RAEDTAVYYCAADTRREWYRDGYWGPPA |
| | | | RYEYDYRGQGTQVTVSS |
| 100 | 1D2 var10 | VHH | EVQLVESGGGLVQPGGSLRLSCAASGRTAS |
| | | | SYVMGWFRQAPGKEREFVSVINWNGDSTY |
| | | | YADSVKGRFTISRDNSKNTLYLQMNSLRAE |
| | | | DTAVYYCAADTRREWYRDGFWGPPARYE |
| | | | YDYRGQGTQVTVSS |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLES

### Example 1. Construction of a Reporter Cell

### Generation of a J.RT3-T3.5 Vγ9Vδ2-TCR Cell

Parental J.RT3-T3.5 cells were co-transfected by electroporation using plasmids encoding CD3 (pExoIN3-CD3, containing a zeocin resistance marker (SEQ ID NO: 137)) and Vγ9Vδ2-TCR receptor (pExoIN2 Vγ9Vδ2-TCR, containing a puromycin resistance marker (SEQ ID NO: 140)). 24 h post transfection transiently transfected cells were stained for Vγ9Vδ2-TCR expression and Vγ9Vδ2-TCR-positive cells were separated by FACS sorting. After 24 h of recovery, the sorted cell population was seeded into soft agar and subjected to antibiotic selection. Outgrowing cells were transferred to standard culture condition and expanded, after which stable cells were stained with specific antibodies for the TCR-Vδ2-chain (TCR Vδ2-FITC, clone IMMU 389, #IM1464, Beckman Coulter), the TCR-Vγ9-chain (PE anti-human TCR Vγ9, Mouse IgG1, kappa, Clone: B3, #BLD-331308, Biozol), and for CD3 (APC anti-human CD3, Mouse IgG2a, kappa, clone: OKT3, #BLD-317318, Biozol), and analyzed for target expression by flow cytometry, resulting in isolation of CD3+Vγ9Vδ2TCR-expressing cells. Parental cells served as control. The stable CD3+Vγ9Vδ2-TCR-expressing cells were enriched by flow cytometry based cell sorting to remove any non-expressing cells and to enrich the fluorescent cell population for high expressing cells.

### Generation of a J.RT3-T3.5 Vg9Vd2-TCR Cell Pool Recombinant for Luciferase

The enriched CD3+Vγ9Vδ2TCR-expressing cells were transfected by electroporation with a plasmid containing a luciferase coding sequence under the control of an NFAT response element, and a hygromycin resistance gene (pGL4.30[luc2P/NFAT-RE/Hygro] Vector (Promega,Cat. #E8481)). 24 h post transfection, transiently transfected cells were stained for Vγ9Vδ2-TCR expression, and Vγ9Vδ2-TCR-positive cells were separated by FACS sorting in order to increase the possibility of co-expression of Vγ9Vd2-TCR and NFAT-RE-Luc. For antibiotic selection, sorted cells were seeded into soft agar and subjected to three different hygromycin concentrations (150 μg/mL, 300 μg/mL and 450 μg/mL hygromycin). In addition, conventional pool generation was started with application of 300 μg/mL hygromycin for selection. Outgrowing cells were tested for luciferase activity upon stimulation (4 hours) with a Ionomycin/PMA mixture (50 ng/mL PMA and 500 ng/mL ionomycin). Parental cells served as negative control, a stable Jurkat-NFAT control cell line served as positive control. Four hours post stimulation a significant luminescence signal could be detected.

### Generation of Clonal J.RT3-T3.5-Vg9Vd2 NFAT-RE Luc Cell Lines

For the generation of clonal J.RT3-T3.5 cell lines stably expressing Vg9Vd2-TCR and NFAT-RE_Luc, single cell cloning was performed by limiting dilution with cells from the stable J.RT3-T3.5-Vg9Vd2_NFAT-RE_Luc cell pool. Cells were single deposited into wells of a 96-well plate. Outgrowing clones were transferred to higher culture formats, expanded, and cryopreserved. After thawing and expansion, twenty-four clones were analyzed for luciferase activity and for Vγ9Vδ2-TCR expression, and the highest expressing clones were preserved.

### Example 2. Protocol for Reporter Cell Assay

This example outlines the protocol used to test a bispecific binding compound using an assay comprising the Jurkat cell (J.RT3-T3.5)-based reporter cell expressing Vγ9Vδ2-TCR generated according to Example 1 (a.k.a., reporter cell), and a PSMA expressing target cell (LNCaP) (herein referred to as target cell).

50 μL of RPMI full medium (RPMI 1640, 10% fetal bovine serum (FBS), 1% pen/strep [penicillin/streptomycin (10,000 U/mL)]) containing 1×10⁵ reporter and 5×10⁴ target cells (2:1 ratio) were added to the wells of a microtiter plate. 50 μL of the bispecific binding compound (having SEQ ID NOS:5, 2, 14, and 16 in which X₂=Y, and SEQ ID NOS:41-44) was added to each well at a desired concentration and the plate incubated for 18 hours at 37° C. and 5% CO₂. Following incubation, 100 μL of ONE-Glo™ luciferase assay reagent (Promega) was added, and the plate incubated in the dark at room temperature (RT) for 15 minutes. Luminescence was then measured using a SpektraMax® M3 plate reader.

### Example 3. Assay Validity Criteria

To determine the potency of the bispecific compound of Example 2, the luminescence values obtained from the plate reader were analyzed with the SoftMax Pro software version 7.1 using a 4PL-fit and “group blank” value subtraction. For confirming the validity of the assay, the parameters listed in in the table below were assessed:

| Acceptance criteria | Acceptance criteria values |
|---|---|
| Raw data background signal | ≤20 Lml RU |
| (medium plus cells without | |
| compound) | |
| Signal/Background ratio | 4x higher than the background for the |
| | top 9 dilutions |
| CV % (Coefficient of Variation) | ≤20% for the top 8 dilutions |
| Goodness of Fit Test (R²) | ≥0.98 |
| EC₅₀ for the bispecific compound | [0.05 nM] ± 25% |
| of Example 2, Batch 54-PS-PBG | |
| Compound samples need to pass | ≥0.8 |
| F-test for parallelism | |

### Example 4. Relative Potency Assessment

If the assay was deemed as valid, additional relative potency analysis was performed. To assess if a constrained fit could be applied, dose response curves were analyzed for their “equality” or parallelism using an F-test. For this test, a value of ≥0.8 was deemed as acceptable. If the outcome was valid, a constrained curve fit/global-fit (PLA) was conducted using the SoftMax Pro version 7.1 software. This allowed for receipt of values for the relative potencies (0.8 (80%)-1.2 (120%)) in comparison to a reference control set as a standard, i.e., EC₅₀ for the bispecific compound of Example 2, batch 54-PS-PBG. Parallelism of the dose-response curves relative to the control was a sample acceptance criterion. If the curves were not parallel, the run was not accepted.

### Example 5. Stability Study

This example demonstrates use of a reporter cell assay of the disclosure to measure the stability of a TDbAb stored under various conditions.

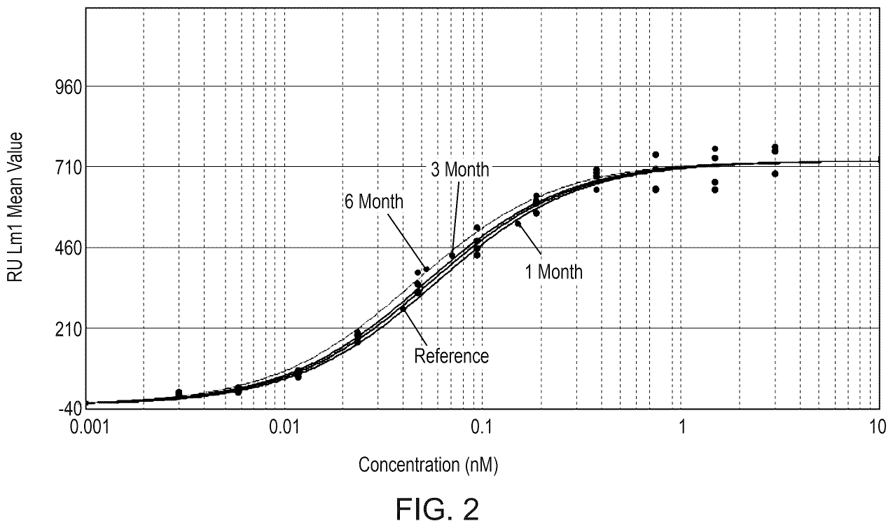

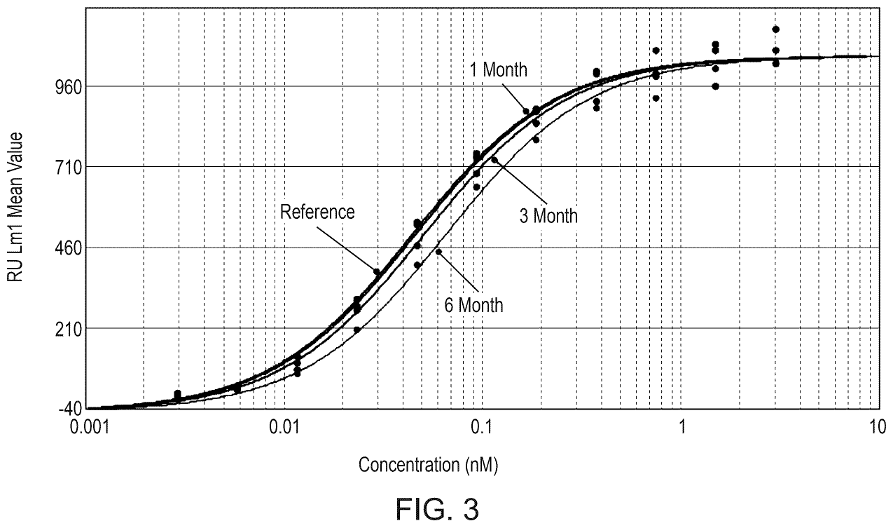

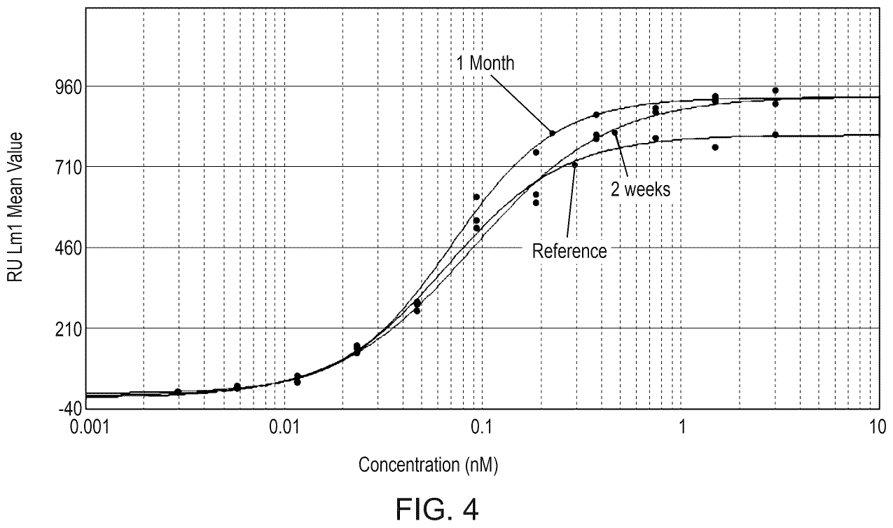

A TDbAb specific for prostate-specific membrane antigen (PSMA) and the TCR delta (δ)2 chain was stored at 5° C. or 20° C. for 1, 3 or 6 months, or at 40° C. for 2 weeks or one month. Following storage, the TDbAb was tested for its ability to activate T cells in the presence of a PSMA expressing target cell, using the protocol described in Example 2. The results are shown in FIGS. 2-4. FIG. 2 shows the estimated relative potency of the TDbAb after storage at 5° C. for various periods of time. FIG. 3 shows the estimated relative potency of the TDbAb after storage at 20° C. for various periods of time. FIG. 4 shows estimated relative potency of the TDbAb when stored at 40° C. for various periods of time.

### Example 6. Detection of Immobilized Antigen

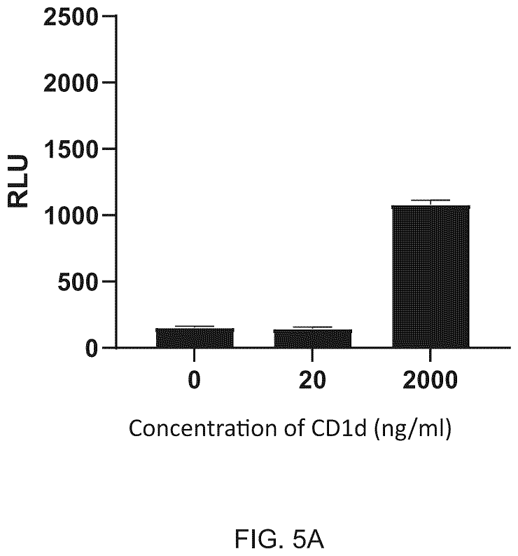

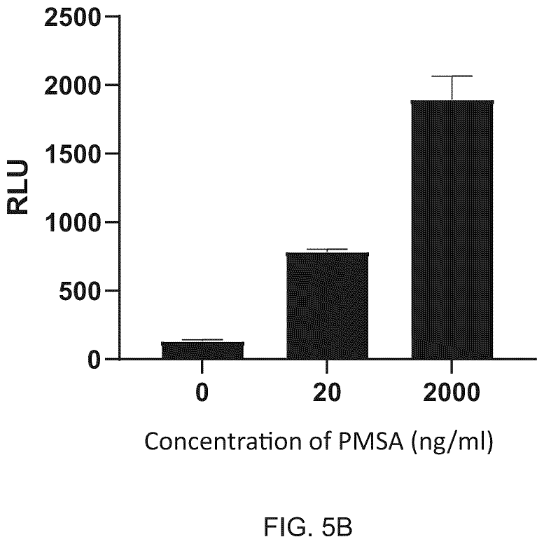

Wells of polystyrene plates were coated with varying concentrations of either CD1d or PSMA, and the plates incubated overnight. The next day, the immobilized antigen was detected using a TDbAb specific for each antigen. TDbAb binding to the immobilized antigen was detected by the addition of the reporter cell line and detection of the luciferase signal. The results are shown in FIG. 5A (CD1d) and FIG. 5B (PSMA).

### Example 7. Generation of Knock-Out Reporter Cell Lines

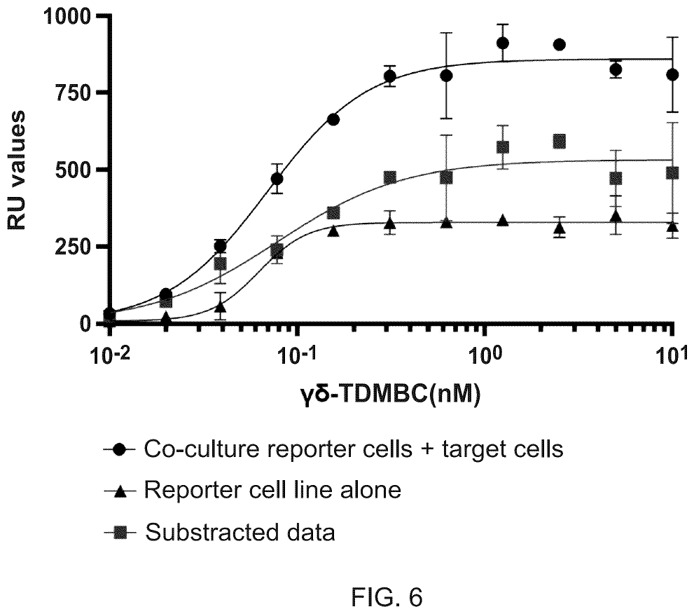

As described herein, expression of the target antigen on the reporter cell line can result in a background luciferase signal with the reporter cell is incubated with a γδ-TDMBC in the absence of a target cell. See e.g., FIG. 6. As shown, co-culture of the reporter cells with target cells in the presence of a γδ-TDMBC resulted in a luciferase signal (circles), indicating activation of the reporter cell by the γδ-TDMBC. However, incubation of the γδ-TDMBC with the reporter cell in the absence of target cells (triangles) resulted in a background luciferase signal. By subtracting the background signal from the signal generated with the presence of the target cells, one can determine the on-target activation of the reporter cell (squares).

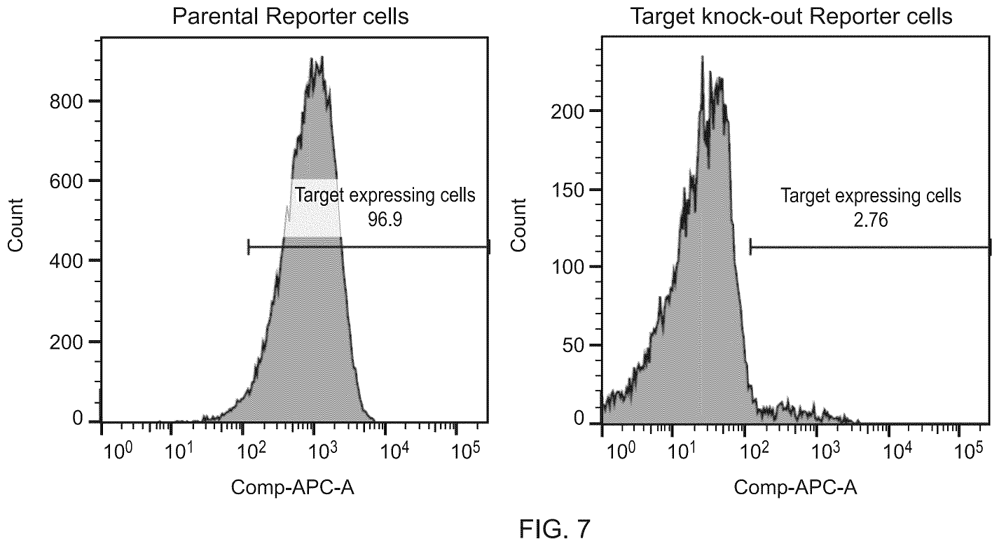

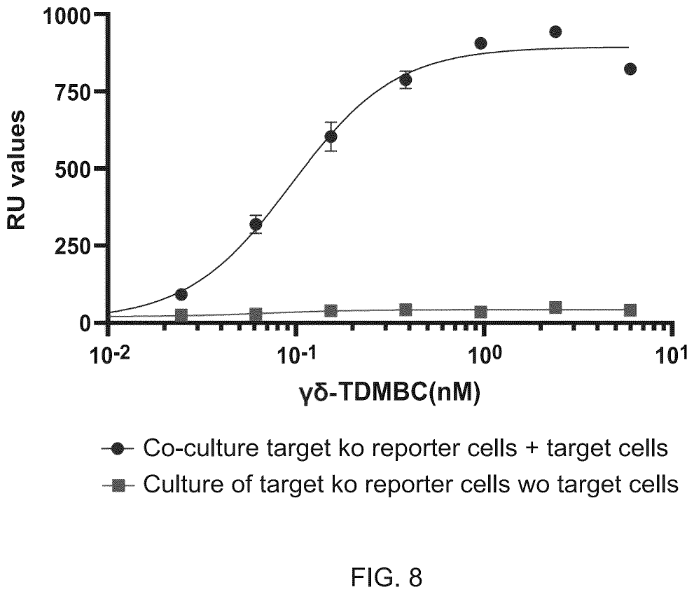

Knock-out reporter cells were generated to eliminate expression of the target antigen on the reporter cell and mitigate the background signal resulting from γδ-TDMBC engagement of the target antigen on the reporter cell. FIG. 7 shows successful knock-out of the CD1d target antigen in the reporter cells. Further, as shown in FIG. 8, elimination of CD1d in the reporter cells also eliminated the background signal seen in the absence of target cells.

1. A method of detecting γδ T cell receptor (γδTCR)-mediated reporter cell activation by a γδ T cell receptor (TCR)-dependent multispecific binding compound (γδ-TDMBC), wherein the γδ-TDMBC comprises a target antigen binding portion and a γδ TCR binding portion, the method comprising:
a) contacting the γδ-TDMBC with:
i) a population of cells comprising a reporter cell that expresses a γδ TCR, and that comprises a reporter gene responsive to activation of the reporter cell; and,
ii) the target antigen; and,
b) detecting expression of the reporter gene,
wherein expression of the reporter gene indicates γδ TCR-mediated activation of the reporter cell.

2.-6. (canceled)

7. The method of claim 1, wherein the reporter cell is a reporter T cell.

8. (canceled)

9. The method of claim 1, wherein the γδ TCR expressed by the reporter cell is a γ9δ2 TCR.

10. The method of claim 1, wherein the reporter cell comprises one or more exogenous nucleic acid molecules encoding the TCR γ chain and/or the TCR δ chains.

11. The method of claim 10, wherein the one or more exogenous nucleic acid molecules are stably integrated into the genome of the reporter cell.

12.-15. (canceled)

16. The method of claim 11, wherein the reporter gene comprises a nucleic acid molecule comprising a nucleotide sequence encoding a reporter protein operably linked to a promoter that is responsive to activation of the reporter cell, and one or more response elements operably linked to the promoter.

17.-20. (canceled)

21. The method of 1, wherein the reporter gene encodes a reporter protein.

22. The method of claim 21, wherein the reporter protein is a fluorescent protein, a luminescent protein, a chemiluminescent protein, or an enzyme.

23. (canceled)

24. (canceled)

25. (canceled)

26. The method of claim 1, wherein the target antigen is a cancer-associated antigen or a tumor-associated antigen.

27. The method of claim 26, wherein the target antigen is selected from the group consisting of Nectin-4, PSMA, CD1d, CD40, EGFR, and CD123.

28.-30. (canceled)

31. The method of claim 1, wherein the γδ-TDMBC is a bispecific antibody.

32. The method of claim 31, wherein the γδ-TDMBC comprises a first single chain domain comprising CDR1, comprising or consisting of SEQ ID NO:5 in which X₁ is G or S, CDR2, comprising or consisting of SEQ ID NO:2, and/or CDR3, comprising or consisting of SEQ ID NO: 14 in which X₂ can be any amino acid and X₃ is not R.

33. (canceled)

34. The method of claim 32, wherein the γδ-TDMBC comprises a second single chain domain comprising:
a. CDR1, comprising or consisting of SEQ ID NO:45, CDR2, comprising or consisting of SEQ ID NO:46, and/or CDR3, comprising or consisting of SEQ ID NO:47;
b. CDR1, comprising or consisting of SEQ ID NO:49, CDR2, comprising or consisting of SEQ ID NO:50, and/or CDR3, comprising or consisting of SEQ ID NO:51;
c. CDR1, comprising or consisting of SEQ ID NO:41, CDR2, comprising or consisting of SEQ ID NO:42, and/or CDR3, comprising or consisting of SEQ ID NO:43;
d. CDR1, comprising or consisting of SEQ ID NO:37, wherein X₄ is G or S, CDR2, comprising or consisting of SEQ ID NO:38, wherein X₅ is A or T, and/or CDR3, comprising or consisting of SEQ ID NO:39, wherein X₆ is Y or F;
e. CDR1, comprising or consisting of SEQ ID NO:53, CDR2, comprising or consisting of SEQ ID NO:54, and/or CDR3, comprising or consisting of SEQ ID NO:55;
f. CDR1, comprising or consisting of SEQ ID NO:57, CDR2, comprising or consisting of SEQ ID NO:58, and/or CDR3, comprising or consisting of SEQ ID NO:59;
g. CDR1, comprising or consisting of SEQ ID NO:61, CDR2, comprising or consisting of SEQ ID NO:62, and/or CDR3, comprising or consisting of SEQ ID NO:63;
h. CDR1, comprising or consisting of SEQ ID NO:65, CDR2, comprising or consisting of SEQ ID NO:66, and/or CDR3, comprising or consisting of SEQ ID NO:67;
i. CDR1, comprising or consisting of SEQ ID NO:69, CDR2, comprising or consisting of SEQ ID NO:70, and/or CDR3, comprising or consisting of SEQ ID NO:71;
j. CDR1, comprising or consisting of SEQ ID NO:73, CDR2, comprising or consisting of SEQ ID NO:74, and/or CDR3, comprising or consisting of SEQ ID NO:75; or
k. CDR1, comprising or consisting of SEQ ID NO:77, CDR2, comprising or consisting of SEQ ID NO:78, and/or CDR3, comprising or consisting of SEQ ID NO:79.

35. The method of claim 34, wherein the second single-domain antibody comprises, or consists of, SEQ ID NO:48, SEQ ID NO:52, SEQ ID NO:44, SEQ ID NO:40, SEQ ID NO: 56, SEQ ID NO: 60, SEQ ID NO: 64, SEQ ID NO: 68, SEQ ID NO: 72, SEQ ID NO: 76, or SEQ ID NO: 80.

36. (canceled)

37. The method of claim 1, wherein the reporter cell comprises one or more knockout mutations in within a TCR α chain locus and/or a TCR β chain locus, or within genetic elements controlling expression of either or both loci.

38. The method of claim 1, wherein the reporter cell does not express the target antigen.

39.-61. (canceled)


SEQUENCE LISTING (140 sequences)

SEQ ID NO: 1 - protein, 5 aa, synthetic construct
NYAMG

SEQ ID NO: 2 - protein, 17 aa, synthetic construct
AISWSGGSTSYADSVKG

SEQ ID NO: 3 - protein, 21 aa, synthetic construct
QFSGADYGFGRLGIRGYEYDY

SEQ ID NO: 4 - protein, 130 aa, synthetic construct
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVSS

SEQ ID NO: 5 - protein, 5 aa, synthetic construct
  VARIANT 5: note = S or G
NYAMX

SEQ ID NO: 6 - protein, 17 aa, synthetic construct
AISWSGGSTSYADSVKG

SEQ ID NO: 7 - protein, 21 aa, synthetic construct
QFSGADYGFGRLGIRGYEYDY

SEQ ID NO: 8 - protein, 5 aa, synthetic construct
NYAMS

SEQ ID NO: 9 - protein, 17 aa, synthetic construct
AISWSGGSTSYADSVKG

SEQ ID NO: 10 - protein, 21 aa, synthetic construct
QFSGADYGFGRLGIRGYEYDY

SEQ ID NO: 11 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADYGFGRLGIRGYEYDYWGQGTQVTVSS

SEQ ID NO: 12 - protein, 5 aa, synthetic construct
  VARIANT 5: note = S or G
NYAMX

SEQ ID NO: 13 - protein, 17 aa, synthetic construct
AISWSGGSTSYADSVKG

SEQ ID NO: 14 - protein, 21 aa, synthetic construct
  VARIANT 7: note = any amino acid
  VARIANT 11: note = any amino acid except R
QFSGADXGFGXLGIRGYEYDY

SEQ ID NO: 15 - protein, 17 aa, synthetic construct
  VARIANT 7: note = any amino acid
  VARIANT 11: note = any amino acid except R
QFSGADXGFGXGYEYDY

SEQ ID NO: 16 - protein, 130 aa, synthetic construct
  VARIANT 35: note = S or G
  VARIANT 105: note = any amino acid
  VARIANT 109: note = any amino acid except R
EVQLLESGGGSVQPGGSLRLSCAASGRPFSNYAMXWFRQAPGKEREFVSAISWSGGSTSYADSVKGRFTISRDNSKNTLYLQMNSLRAEDTAVYYCAAQFSGADXGFGXLGIRGYEYDYWGQGTQVTVSS

SEQ ID NO: 17 - protein, 21 aa, synthetic construct
QFSGADFGFGALGIRGYEYDY

SEQ ID NO: 18 - protein, 21 aa, synthetic construct
QFSGADSGFGALGIRGYEYDY

SEQ ID NO: 19 - protein, 21 aa, synthetic construct
QFSGADFGFGKLGIRGYEYDY

SEQ ID NO: 20 - protein, 21 aa, synthetic construct
QFSGADSGFGKLGIRGYEYDY

SEQ ID NO: 21 - protein, 17 aa, synthetic construct
QFSGADFGFGAGYEYDY

SEQ ID NO: 22 - protein, 17 aa, synthetic construct
QFSGADSGFGAGYEYDY

SEQ ID NO: 23 - protein, 17 aa, synthetic construct
QFSGADFGFGKGYEYDY

SEQ ID NO: 24 - protein, 17 aa, synthetic construct
QFSGADSGFGKGYEYDY

SEQ ID NO: 25 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGALGIRGYEYDYWGQGTQVTVSS

SEQ ID NO: 26 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGALGIRGYEYDYWGQGTQVTVSS

SEQ ID NO: 27 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGKLGIRGYEYDYWGQGTQVTVSS

SEQ ID NO: 28 - protein, 130 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGKLGIRGYEYDYWGQGTQVTVSS

SEQ ID NO: 29 - protein, 126 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGAGYEYDYWGQGTQVTVSS

SEQ ID NO: 30 - protein, 126 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGAGYEYDYWGQGTQVTVSS

SEQ ID NO: 31 - protein, 126 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGKGYEYDYWGQGTQVTVSS

SEQ ID NO: 32 - protein, 126 aa, synthetic construct
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGKGYEYDYWGQGTQVTVSS

SEQ ID NO: 33 - protein, 5 aa, synthetic construct
SYGMG

SEQ ID NO: 34 - protein, 17 aa, synthetic construct
GISWRGDSTGYADSVKG

SEQ ID NO: 35 - protein, 15 aa, synthetic construct
AAGSAWYGTLYEYDY

SEQ ID NO: 36 - protein, 124 aa, synthetic construct
EVQLVESGGGSVQPGGSLRLSCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTLVTVSS

SEQ ID NO: 37 - protein, 10 aa, synthetic construct
  VARIANT 10: note = G or S
GRTASSYVMX

SEQ ID NO: 38 - protein, 17 aa, synthetic construct
  VARIANT 12: note = A or T
VINWNGDSTYYXDSVKG

SEQ ID NO: 39 - protein, 22 aa, synthetic construct
  VARIANT 11: note = Y or F
DTRREWYRDGXWGPPARYEYDY

SEQ ID NO: 40 - protein, 131 aa, synthetic construct
EVQLVESGGGLVQAGGSLRLSCAASGRTASSYVMGWFRQAPGKEREFVAVINWNGDSTYYTDSVKGRFAISRDNAKNTVYLQMNSLKPEDTAVYYCAADTRREWYRDGYWGPPARYEYDYRGQGTQVTVSS

SEQ ID NO: 41 - protein, 10 aa, synthetic construct
RFMISEYSMH

SEQ ID NO: 42 - protein, 16 aa, synthetic construct
TINPAGTTDYADSVKG

SEQ ID NO: 43 - protein, 5 aa, synthetic construct
DGYGY

SEQ ID NO: 44 - protein, 111 aa, synthetic construct
EVQLVESGGGLVQPGGSLRLSCAASRFMISEYSMHWVRQAPGKGLEWVSTINPAGTTDYADSVKGRFTISRDNAKNTLYLQMNSLRAEDTAVYYCDGYGYRGQGTQVTVSS

SEQ ID NO: 45 - protein, 5 aa, synthetic construct
DNVMG

SEQ ID NO: 46 - protein, 16 aa, synthetic construct
TIRTGGSTNYADSVKG

SEQ ID NO: 47 - protein, 11 aa, synthetic construct
TIPVPSTPYDY

SEQ ID NO: 48 - protein, 119 aa, synthetic construct
QVQLVESGGGLVQAGGSLRLSCAASGSMFSDNVMGWYRQAPGKQRELVATIRTGGSTNYADSVKGRFTISRDNAKNTVYLQMNSLKPEDTAVYYCRHTIPVPSTPYDYWGQGTQVTVSS

SEQ ID NO: 49 - protein, 5 aa, synthetic construct
RSAMG

SEQ ID NO: 50 - protein, 17 aa, synthetic construct
AIGTRGGSTKYADSVKG

SEQ ID NO: 51 - protein, 17 aa, synthetic construct
RGPGYPSAAIFQDEYHY

SEQ ID NO: 52 - protein, 126 aa, synthetic construct
QVQLQESGGGLVQAGGSLRLSCAASGRTFGRSAMGWFRQAPGKEREFVAAIGTRGGSTKYADSVKGRFTISTDNASNTVYLQMDSLKPEDTAVYRCAVRGPGYPSAAIFQDEYHYWGQGTQVTVSS

SEQ ID NO: 53 - protein, 5 aa, synthetic construct
INLMG

SEQ ID NO: 54 - protein, 16 aa, synthetic construct
SISPGGSVRYADSVKG

SEQ ID NO: 55 - protein, 10 aa, synthetic construct
ESERTYYFDS

SEQ ID NO: 56 - protein, 118 aa, synthetic construct
EVQLVESGGGLVQAGGSLRLSCAASGSISSINLMGWFRQAPAKQRELVTSISPGGSVRYADSVKGRFTISRDVAKNTVDLQMNSLKPEDTAVYYCAAESERTYYFDSWGQGTQVTVSS

SEQ ID NO: 57 - protein, 16 aa, synthetic construct
TITRGGSTNYADSVKG

SEQ ID NO: 58 - protein, 11 aa, synthetic construct
VEPSGMGWRDY

SEQ ID NO: 59 - protein, 30 aa, synthetic construct
EVQLVESGGGLVQAGGSLRLSCAASGSISS

SEQ ID NO: 60 - protein, 119 aa, synthetic construct
EVQLVESGGGLVQAGGSLRLSCAASGSISSINLMGWHRQAPGKQRELVATITRGGSTNYADSVKGRFTISRDNGKNTVYLQMNSLKPEDTAAYYCVDVEPSGMGWRDYWGQGTQVTVSS

SEQ ID NO: 61 - protein, 5 aa, synthetic construct
LNIMG

SEQ ID NO: 62 - protein, 16 aa, synthetic construct
TITTGGSTNYADSVRG

SEQ ID NO: 63 - protein, 10 aa, synthetic construct
ELVRRGPTTY

SEQ ID NO: 64 - protein, 118 aa, synthetic construct
EVQLVESGGGLVQAGGSLRLSCAASRSTFSLNIMGWYRQAPGKQREYVATITTGGSTNYADSVRGRFTISRDNAEDTVYLQMNSLKPEDTAVYYCTAELVRRGPTTYWGRGTQVTVSS

SEQ ID NO: 65 - protein, 5 aa, synthetic construct
LNIMG

SEQ ID NO: 66 - protein, 16 aa, synthetic construct
TITSGGSTNYADSVRG

SEQ ID NO: 67 - protein, 10 aa, synthetic construct
ELVRRGPTTY

SEQ ID NO: 68 - protein, 118 aa, synthetic construct
EVQLVESGGGLVQAGGSLRLSCAASRSTFSLNIMGWYRQAPGKQREYVATITSGGSTNYADSVRGRFTISRDNAENTVYLQMDSLKPEDTAVYYCTAELVRRGPTTYWGRGTQVTVSS

SEQ ID NO: 69 - protein, 5 aa, synthetic construct
RLTMG

SEQ ID NO: 70 - protein, 17 aa, synthetic construct
RVYASGGLTDYADSVKG

SEQ ID NO: 71 - protein, 15 aa, synthetic construct
GLWADMRTMTSTRGY

SEQ ID NO: 72 - protein, 124 aa, synthetic construct
EVQLVESGGALVQAGGSLRLSCSVSGLTASRLTMGWFRQAPGKEREFVARVYASGGLTDYADSVKGRFTISRDNAKNTVYLQMNSLEPEDTAVYYCVAGLWADMRTMTSTRGYWGQGTQVTVSS

SEQ ID NO: 73 - protein, 5 aa, synthetic construct
INLMG

SEQ ID NO: 74 - protein, 16 aa, synthetic construct
SITRGGSTWYVDSVKG

SEQ ID NO: 75 - protein, 10 aa, synthetic construct
ESIGSYTFDR

SEQ ID NO: 76 - protein, 118 aa, synthetic construct
EVQLVESGGGLVQAGGSLRLSCAASGSIFDINLMGWYRQAPGKLRELVASITRGGSTWYVDSVKGRFIISRDSAKNTVYLQMNSLKPGDTAVYYCAAESIGSYTFDRWGQGTQVTVSS

SEQ ID NO: 77 - protein, 5 aa, synthetic construct
INVMG

SEQ ID NO: 78 - protein, 16 aa, synthetic construct
GITSGGSRRLADSVKG

SEQ ID NO: 79 - protein, 9 aa, synthetic construct
EAVVGDEPY

SEQ ID NO: 80 - protein, 117 aa, synthetic construct
EVQLVESGGGLVQAGGFLRLSCAASRSVFRINVMGWYRQAPGKQRELVAGITSGGSRRLADSVKGRFTISRDNAKNTVYLQMNSLKPEDTAVYYCFAEAVVGDEPYWGQGTQVTVSS

SEQ ID NO: 81 - protein, 5 aa, synthetic construct
GGGGS

SEQ ID NO: 82 - protein, 14 aa, synthetic construct
AAASDKTHTCPPCP

SEQ ID NO: 83 - protein, 231 aa, synthetic construct
  VARIANT 231: note = K or may be absent
AAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 84 - protein, 231 aa, synthetic construct
  VARIANT 231: note = K or may be absent
AAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 85 - protein, 355 aa, synthetic construct
  VARIANT 355: note = K or may be absent
EVQLVESGGGSVQPGGSLRLSCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTLVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 86 - protein, 362 aa, synthetic construct
  VARIANT 362: note = K or may be absent
EVQLVESGGGLVQAGGSLRLSCAASGRTASSYVMGWFRQAPGKEREFVAVINWNGDSTYYTDSVKGRFAISRDNAKNTVYLQMNSLKPEDTAVYYCAADTRREWYRDGYWGPPARYEYDYRGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGX

SEQ ID NO: 87 - protein, 361 aa, synthetic construct
  VARIANT 361: note = K or may be absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGALGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 88 - protein, 361 aa, synthetic construct
  VARIANT 361: note = K or may be absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGALGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 89 - protein, 361 aa, synthetic construct
  VARIANT 361: note = K or may be absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADFGFGKLGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 90 - protein, 361 aa, synthetic construct
  VARIANT 361: note = K or may be absent
EVQLLESGGGSVQPGGSLRLSCAASGRPFSNYAMSWFRQAPGKEREFVSAISWSGGSTSYADSVKGRFTISRDNSKNTLYLQMNSLRAEDTAVYYCAAQFSGADSGFGKLGIRGYEYDYWGQGTQVTVSSAAASDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 91 - protein, 131 aa, synthetic construct
EVQLVESGGGLVQPGGSLRLSCAASGRTASSYVMGWFRQAPGKEREFVSVINWNGDSTYYADSVKGRFTISRDNSKNTLYLQMNSLRAEDTAVYYCAADTRREWYRDGYWGPPARYEYDYRGQGTQVTVSS

SEQ ID NO: 92 - protein, 131 aa, synthetic construct
EVQLVESGGGLVQPGGSLRLSCAASGRTASSYVMGWVRQAPGKEREWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 93 - protein, 131 aa, synthetic construct
EVQLVESGGGLVQPGGSLRLSCAASGRTASSYVMSWFRQAPGKEREWVAVINWNGDSTYYADSVKGRFTISRDNSKNTVYLQMNSLRAEDTAVYYCAADTRREWYRDGYWGPPARYEYDYRGQGTLVTVSS

SEQ ID NO: 94 - protein, 131 aa, synthetic construct
EVQLVESGGGVVQPGGSLRLSCAASGRTASSYVMSWFRQAPGKEREWVAVINWNGDSTYYADSVKGRFTISRDNSKNTLYLQMNSLRAEDTAVYYCAADTRREWYRDGYWGPPARYEYDYRGQGTTVTVSS

SEQ ID NO: 95 - protein, 131 aa, synthetic construct
EVQLVESGGGLVQPGGSLRLSCAASGRTASSYVMSWFRQAPGKEREFVAVINWNGDSTYYADSVKGRFTISRDNSKNTVYLQMNSLRAEDTAVYYCAADTRREWYRDGYWGPPARYEYDYRGQGTLVTVSS

SEQ ID NO: 96 - protein, 131 aa, synthetic construct
EVQLVESGGGLVQPGGSLRLSCAASGRTASSYVMGWVRQAPGKGLEWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 97 - protein, 131 aa, synthetic construct
EVQLVESGGGLVQPGGSLRLSCAASGRTASSYVMSWVRQAPGKGLEWVSVINWNGDSTYYADSVKGRFTISRDNSKNTLYLQMNSLRAEDTAVYYCAADTRREWYRDGYWGPPARYEYDYRGQGTLVTVSS

SEQ ID NO: 98 - protein, 131 aa, synthetic construct
EVQLVESGGGLVQPGGSLRLSCAASGRTASSYVMSWFRQAPGKGLEWVAVINWNGDSTYYADSVKGRFTISRDNSKNTVYLQMNSLRAEDTAVYYCAADTRREWYRDGYWGPPARYEYDYRGQGTLVTVSS

SEQ ID NO: 99 - protein, 131 aa, synthetic construct
EVQLVESGGGLVQAGGSLRLSCAASGRTASSYVMGWFRQAPGKEREWVAVINWNGDSTYYTDSVKGRFAISRDNAKNTVYLQMNSLRAEDTAVYYCAADTRREWYRDGYWGPPARYEYDYRGQGTQVTVSS

SEQ ID NO: 100 - protein, 131 aa, synthetic construct
EVQLVESGGGLVQPGGSLRLSCAASGRTASSYVMGWFRQAPGKEREFVSVINWNGDSTYYADSVKGRFTISRDNSKNTLYLQMNSLRAEDTAVYYCAADTRREWYRDGFWGPPARYEYDYRGQGTQVTVSS

SEQ ID NO: 101 - protein, 5 aa, synthetic construct
RSAMG

SEQ ID NO: 102 - protein, 17 aa, synthetic construct
AIGTRGGSTKYADSVKG

SEQ ID NO: 103 - protein, 17 aa, synthetic construct
RGPGYPSAAIFQDEYHY

SEQ ID NO: 104 - protein, 126 aa, synthetic construct; identical to SEQ ID NO: 52

SEQ ID NO: 105 - protein, 5 aa, synthetic construct
INLMG

SEQ ID NO: 106 - protein, 16 aa, synthetic construct
SISPGGSVRYADSVKG

SEQ ID NO: 107 - protein, 10 aa, synthetic construct
ESERTYYFDS

SEQ ID NO: 108 - protein, 118 aa, synthetic construct; identical to SEQ ID NO: 56

SEQ ID NO: 109 - protein, 16 aa, synthetic construct
TITRGGSTNYADSVKG

SEQ ID NO: 110 - protein, 11 aa, synthetic construct
VEPSGMGWRDY

SEQ ID NO: 111 - protein, 30 aa, synthetic construct; identical to SEQ ID NO: 59

SEQ ID NO: 112 - protein, 119 aa, synthetic construct; identical to SEQ ID NO: 60

SEQ ID NO: 113 - protein, 5 aa, synthetic construct
LNIMG

SEQ ID NO: 114 - protein, 16 aa, synthetic construct
TITTGGSTNYADSVRG

SEQ ID NO: 115 - protein, 10 aa, synthetic construct
ELVRRGPTTY

SEQ ID NO: 116 - protein, 118 aa, synthetic construct; identical to SEQ ID NO: 64

SEQ ID NO: 117 - protein, 5 aa, synthetic construct
LNIMG

SEQ ID NO: 118 - protein, 16 aa, synthetic construct
TITSGGSTNYADSVRG

SEQ ID NO: 119 - protein, 10 aa, synthetic construct
ELVRRGPTTY

SEQ ID NO: 120 - protein, 118 aa, synthetic construct; identical to SEQ ID NO: 68

SEQ ID NO: 121 - protein, 5 aa, synthetic construct
RLTMG

SEQ ID NO: 122 - protein, 17 aa, synthetic construct
RVYASGGLTDYADSVKG

SEQ ID NO: 123 - protein, 15 aa, synthetic construct
GLWADMRTMTSTRGY

SEQ ID NO: 124 - protein, 124 aa, synthetic construct; identical to SEQ ID NO: 72

SEQ ID NO: 125 - protein, 5 aa, synthetic construct
INLMG

SEQ ID NO: 126 - protein, 16 aa, synthetic construct
SITRGGSTWYVDSVKG

SEQ ID NO: 127 - protein, 10 aa, synthetic construct
ESIGSYTFDR

SEQ ID NO: 128 - protein, 118 aa, synthetic construct; identical to SEQ ID NO: 76

SEQ ID NO: 129 - protein, 5 aa, synthetic construct
INVMG

SEQ ID NO: 130 - protein, 16 aa, synthetic construct
GITSGGSRRLADSVKG

SEQ ID NO: 131 - protein, 9 aa, synthetic construct
EAVVGDEPY

SEQ ID NO: 132 - protein, 117 aa, synthetic construct; identical to SEQ ID NO: 80

SEQ ID NO: 133 - protein, 150 aa, Homo sapiens
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 134 - protein, 160 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 135 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 136 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 137 - protein, 792 aa, synthetic construct
  REGION 1..792: note = human T-cell surface glycoprotein CD3 linked sequence (delta-F2A-gamma-T2A-epsilon-P2A-zeta)
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKGSGVKQTLNFDLLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNGSGEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGSGATNFSLLKQAGDVEENPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 138 - protein, 242 aa, Homo sapiens
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDN

SEQ ID NO: 139 - protein, 229 aa, Homo sapiens
AIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKS

SEQ ID NO: 140 - protein, 629 aa, synthetic construct
MLSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGATNFSLLKQAGDVEENPGPMQRISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDTLGMGGEYTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL